(19) United States
(12) Patent Application Publication
(10) Pub. No.: US 2011/0014137 A1
(43) Pub. Date: Jan. 20, 2011

(54) METHODS FOR PROTECTING THE SKIN FROM RADIATION INSULTS

(75) Inventors: Paul Talalay, Baltimore, MD (US); Albena T. Dinkova-Kostova, Dundee (GB)

Correspondence Address: EDWARDS ANGELL PALMER & DODGE LLP, P.O. BOX 55874, BOSTON, MA, 02205, US

(73) Assignee: THE JOHNS HOPKINS UNIVERSITY, Baltimore, MD (US)

(21) Appl. No.: 12/761,551

(22) Filed: Apr. 16, 2010

Related U.S. Application Data
(63) Continuation of application No. PCT/US2008/011792, filed on Oct. 16, 2008.
(60) Provisional application No. 60/960,849, filed on Oct. 16, 2007.

(51) Int. Cl. A61K 31/26; A61K 31/7028; A61Q 17/04; A61P 17/16; A61P 7/10; A61P 29/00; A61P 1/04; A61P 17/00; A61P 35/00

(52) U.S. Cl. 424/59; 514/514; 514/24

(57) ABSTRACT

The present invention relates to methods and compositions for the protection of skin and mucous membranes from undesirable side effects of ionizing radiation in a patient undergoing ionizing radiation therapy. In particular, the application describes compositions and methods comprising the topical use of Nrf2 inducers.

This application is a Continuation of Application PCT/US2008/011792 (WO2009/051739) filed on Oct. 16, 2008, which claims the benefit of U.S. Provisional Application 60/960,849 filed on Oct. 16, 2007, both of which applications are expressly incorporated herein by reference in their entirety. The skin is continuously exposed to changes in the external environment, including oxidative insults, heat, cold, UV radiation, injury, and mechanical stresses. The stratum corneum, composed of terminally differentiated keratinocytes, constitutes the natural barrier that prevents loss of water and prevents entry of infectious agents (e.g., bacteria, viruses), small objects (e.g., particles), and a broad variety of water-soluble chemicals.

### BACKGROUND OF THE INVENTION

The present invention is directed to methods for protecting the skin and mucous membranes from external insults, including radiations.

### SUMMARY OF THE INVENTION

Further to this object, the invention provides methods to protect the skin and mucous membranes in a patient undergoing ionizing radiation treatment comprising topically administering to the area of the patient's body exposed to ionizing radiation and surrounding areas a composition comprising a therapeutically effective amount of an Nrf2 inducer. The patient to be treated may suffer from short-term or long-term effects of ionizing radiation treatment. In one aspect of the invention, the patient may be affected by acute erythema, skin irritation, inflammation, edema, desquamation, necrosis of the skin, soreness and ulceration in the mouth, pain, fibrosis, telangiectasia, xerostomia, xerophthalmia, dryness and irritation of the vaginal or rectal mucosa, melanoma, breast cancer, stomach cancer, lung cancer, or thyroid disorders. In another aspect of the invention, the patient to be treated may have no symptoms.

In a further embodiment, the method to protect the skin and mucous membranes in a patient undergoing ionizing radiation treatment comprises topically administering to the area of the patient's body exposed to ionizing radiation and surrounding areas a composition comprising a therapeutically effective amount of a phase II enzyme inducer. In one embodiment, the phase II inducer is an isothiocyanate. In a preferred embodiment the phase II enzyme inducer is sulforaphane. In another preferred embodiment, the phase II enzyme inducer is a sulforaphane synthetic analogue. Sulforaphane analogs can be selected from the group consisting of 6-isothiocyanato-2-hexanone, exo-2-acetyl-6-isothiocyanatonorbornane, exo-2-isothiocyanato-6-methylsulfonylnorbornane, 6-isothiocyanato-2-hexanol, 1-isothiocyanato-4-dimethylphosphonylbutane, exo-2-(1′-hydroxyethyl)-5-isothiocyanatonorbornane, exo-2-acetyl-5-isothiocyanatonorbornane, 1-isothiocyanato-5-methylsulfonylpentane, cis-3-(methylsulfonyl)cyclohexylmethylisothiocyanate and trans-3-(methylsulfonyl)cyclohexylmethylisothiocyanate.

In yet another embodiment, the Nrf2 inducer is a glucosinolate. In an additional embodiment, the composition is administered to the patient prior to, during or after ionizing radiation therapy.

In an additional embodiment, the present invention provides a composition for topical application to the skin comprising a therapeutically effective amount of an Nrf2 inducer and a vehicle suitable for delivery. Vehicles suitable for topical delivery of the Nrf2 inducer include jojoba oil and evening primrose oil.

Preferably, the Nrf2 inducer in the composition is a phase II enzyme inducer. More preferably, the phase II inducer is an isothiocyanate. Even more preferably, the phase II enzyme inducer is sulforaphane or a sulforaphane synthetic analogue. Sulforaphane analogs can be selected from the group consisting of 6-isothiocyanato-2-hexanone, exo-2-acetyl-6-isothiocyanatonorbornane, exo-2-isothiocyanato-6-methylsulfonylnorbornane, 6-isothiocyanato-2-hexanol, 1-isothiocyanato-4-dimethylphosphonylbutane, exo-2-(1′-hydroxyethyl)-5-isothiocyanatonorbornane, exo-2-acetyl-5-isothiocyanatonorbornane, 1-isothiocyanato-5-methylsulfonylpentane, cis-3-(methylsulfonyl)cyclohexylmethylisothiocyanate and trans-3-(methylsulfonyl)cyclohexylmethylisothiocyanate.

In another embodiment, the Nrf2 inducer is a glucosinolate. Preferably, the composition for topical administration is in the form of ointment, cream, emulsion, lotion, gel or sunscreen.

The foregoing general description and following brief description of the drawings and the detailed description are exemplary and explanatory and are intended to provide further explanation of the invention as claimed. Other objects, advantages, and novel features will be readily apparent to those skilled in the art from the following detailed description of the invention.

### DESCRIPTION OF THE DRAWINGS

FIG. 1 graphically demonstrates the induction of quinone reductase (NQO1) and elevation of GSH as a function of concentration of sulforaphane in PE murine keratinocytes (A) and human HaCaT keratinocytes (B). Cells (20,000 per well) were plated on 96-well plates and exposed to a series of concentrations of sulforaphane. GSH and NQO1 levels were measured in cell lysates after 24 h and 48 h, respectively. Each data point represents the average of the measurements from 8 different wells. The standard deviation was <5% for all data points.

FIG. 2 provides a graph showing the protection afforded by sulforaphane in PE murine keratinocytes against UVA radiation-generated reactive oxygen intermediates. Cells (50,000 per well) were plated on 24-well plates, treated with 5 μM sulforaphane for 24 h, washed with DPBS, and then exposed to UVA (10 J/cm²). Reactive oxygen intermediates generated by the UV radiation were quantified by the fluorescent probe 2′,7′-dichlorodinitrofluorescein and fluorescence intensity was measured (expressed as a ratio of exposed to non-exposed cells).

FIG. 3 shows the time course of induction of quinone reductase (NQO1) in human skin of healthy human volunteers by single topical application of 100 nmol sulforaphane.

FIG. 4 shows induction of NQO1 in human skin of healthy human volunteers by three repeated topical applications of 50 nmol of sulforaphane at 24 hour intervals.

FIGS. 5A, 5B and 5C show the inhibition caused by sulforaphane on (A) NO production and iNOS mRNA (B) and protein (C) induction in RAW 264.7 cells stimulated with γ-interferon or lipopolysaccharide. Cells were treated with various concentrations of sulforaphane and either IFNγ (10 ng/ml) or lipopolysaccharide (LPS; 3 ng/ml) for 24 h. NO in the medium was measured as nitrite by the Griess reaction (A), and iNOS induction was detected by Northern (B) and Western (C) blotting.

FIGS. 6A and 6B demonstrate the inhibition by sulforaphane of UVB radiation-induced skin carcinogenesis in high-risk mice.

FIG. 7 graphically shows the inhibition of overall tumor burden in high-risk mice by transdermal administration of sulforaphane. Tumor burden is expressed as total volume of all tumors in mm³ divided by the number of animals at risk. Average values±SE are shown. There was a dramatic and highly significant effect (p<0.0027) of concentration (treatment) upon log transformation of tumor volume (ANOVA of concentration using treatment time as a nested variable).

FIG. 8 provides a graph showing the impact of sulforaphane on the multiplicity of small (<1 cm³, white bars) and large tumors (>1 cm³, black bars). Eleven weeks after treatment with protector or vehicle, the tumor incidence in the control group was 100%, and the experiment was terminated. All mice were euthanized on the same day and the tumor size was measured. Low dose, 0.3 μmol sulforaphane, high dose, 1.0 μmol sulforaphane applied daily, 5 times a week, to the backs of the animals.

FIG. 9 provides a graph showing the tumor incidence (percent mice with tumors) in high-risk mice receiving dietary administration of sulforaphane. The control group is depicted as circles, the low dose group is depicted as squares and the high dose group is depicted as triangles. Tumor incidence was reduced by 25% and 35% in the animals receiving low dose and high dose of glucoraphanin, respectively, as compared to the control group.

FIG. 10 provides a graph showing tumor multiplicity (number of tumors per mouse) in high-risk mice receiving dietary administration of sulforaphane. The control group is depicted as circles, the low dose group is depicted as squares and the high dose group is depicted as triangles. Tumor multiplicity was reduced by 47% and 72%, respectively, as compared to the control group.

FIG. 11 provides a graph showing tumor burden (total tumor volume) per mouse in high-risk mice receiving dietary administration of sulforaphane. The control group is depicted as circles, the low dose group is depicted as squares and the high dose group is depicted as triangles. Both low dose and high dose of glucoraphanin treatment resulted in 70% inhibition in the total tumor volume per mouse as compared to the control group.

FIGS. 12A, 12B, 12C, 12D and 12E show the protection of mouse skin provided by sulforaphane and sulforaphane-rich broccoli sprout extracts against edema and inflammatory effects of 311-nm UV radiation. The backs of SKH-1 hairless mice were treated topically with three doses at 24-h intervals of: (i) broccoli sprout extract containing 0.5 μmol of sulforaphane in 50 μl of 80% acetone/20% water (vol/vol) applied to the caudal area, and (ii) solvent applied to the rostral area. The animals received 700 mJ/cm² of 311-nm UV radiation 24 h after the last dose and were euthanized 24 h later, and their dorsal skin was harvested. (A) Fresh frozen 9-μm-thick sections of skin were fixed with paraformaldehyde and stained with H&E. (Scale bar 100 μm). (B) Mice were irradiated with a range of doses of UV radiation and euthanized 24 h later. (C) MPO-specific activity was measured in supernatant fractions of total homogenates prepared from liquid nitrogen-frozen and pulverized dorsal skin, and its protein levels were detected by Western blots with anti-MPO antibody (Hycult Biotechnology, Uden, The Netherlands). Uniformity of protein levels was confirmed by Coomassie blue staining of a parallel gel (data not shown). (D and E) MPO-specific (D) and NQO1-specific (E) activities were measured in supernatant fractions of total skin homogenates from mice treated with solvent (black bars), sulforaphane (gray bars), and broccoli sprout extract (white bars) and are expressed as ratios of each treatment to the non-irradiated control. Average values±SD are shown. Eight animals were used in the control group, and four in each of the treatment groups. Treatment with either sulforaphane or broccoli sprout extract led to equivalent protection against the UV radiation-induced MPO elevation (sulforaphane, P=0.005; broccoli sprout extract, P=0.001), and restoration of the UV radiation decreased NQO1 levels (sulforaphane, P=0.003; broccoli sprout extract, P=0.00001).

FIGS. 13A and 13 B show that the intensity of erythema depends linearly on the dose of UV radiation. (A) Adhesive vinyl templates placed on the back of the chest in the paraspinal regions. The apertures are 2.0-cm diameter and can be individually occluded to provide a range of UV radiation doses. The positions of the small holes at the four corners of each template are marked with a skin marker to locate the templates precisely in the same positions on successive days. (B) Intensity of erythema as a function of UV radiation dose. The erythema values (a*) were measured on 2.0-cm-diameter circles on the back of a male subject immediately before and 24 h after exposure to 100-800 mJ/cm² of 311-nm UV radiation. Two pairs of adjacent spots were assigned to each UV dose. The mean changes in a* values after radiation are shown (filled circles), together with bars indicating the range of the duplicate values. The mean a* value for all 16 spots before radiation was 6.22±1.91 (CV=30.7%). The linear correlation coefficient (r²) of the increment of a* values with respect to UV dose is 0.986.

FIGS. 14A, 14B, 14C and 14 D show the protection of human skin provided by sulforaphane-rich broccoli sprout extracts against erythema caused by 311-nm UV radiation. (A) inhibition of skin erythema development by topical treatment of a male volunteer with a range of sulforaphane doses. The circular 2.0-cm-diameter spots received 100, 200, 400, or 600 nmol sulforaphane as broccoli sprout extract in 25 μl of 80% acetone/20% water on 3 days at 24-h intervals. Control spots received 25 μl of solvent only. Chromometer measurements of a* were obtained 4 days before radiation with 500 mJ/cm² of UV radiation and 24 h after radiation. The 4-day mean a* values for the solvent-treated areas before radiation was 6.70±1.16. Inhibition of erythema formation (%) was calculated from [a*(untreated)−a*(treated)/a*(untreated)]×100. The untreated values (zero dose) were calculated from the increment of two areas that received 25 μl of broccoli sprout extract in 80% acetone/20% water containing 400 nmol of unhydrolyzed glucoraphanin (the inactive glucosinolate precursor of sulforaphane). (B) Photograph of four pairs of spots of individuals (described in A) who received 100, 200, 400, or 600 nmol doses of sulforaphane (as broccoli sprout extract) or solvent only. (C) Effect of topical treatment with sulforaphane-containing broccoli sprout extract on erythema response to a range of doses of UV radiation. With the use of 16-window template, horizontally adjacent pairs of spots were treated with either 200 nmol of sulforaphane in 25 μl of 80% acetone/20% water or solvent alone on 3 successive days at 24-h intervals and 24 h later were radiated with 100-800 mJ/cm² of UV radiation. The increments in a* values for each spot after UV radiation with respect to their 4-day means before UV radiation are plotted as a function of UV dose. The visually determined minimum erythema dose was 600 mJ/cm². (D) Photographs of pairs of broccoli sprout extract- and solvent-treated spots that received 500, 600, or 700 mJ/cm² of UV radiation. The complete set of percent reduction values for this subject are shown in Table 1 (subject 2).

### DETAILED DESCRIPTION

Ionizing radiation therapy or radiotherapy is commonly used for the treatment of malignant tumors. Ionizing radiations may be used to kill cancer cells and shrink tumors in almost every type of solid tumor, including cancers of the brain, breast, cervix, larynx, lung, pancreas, prostate, skin, spine, stomach, uterus and soft tissue sarcomas. Radiation can also be used to treat leukemia and lymphoma. Radiotherapy may be used as a palliative treatment in the absence of a cure for local control of the tumor or symptomatic release, or as a therapeutic treatment to extend the life span of the patient. Total body irradiation is performed prior to bone marrow transplant. In some cases, radiotherapy is used for the treatment of non-malignant conditions, such as trigeminal neuralgia, thyroid eye disease, pterygium and prevention of keloid scar growth or heterotopic ossification. Hyperthermia, or deep tissue heating, is often used in conjunction with radiation to increase the responsiveness of large or advanced tumors to the treatment.

Radiation therapy destroys the cells in the target tissue by damaging their DNA, modifying signal transduction pathways and inducing apoptosis. Ionizing radiation consists of electromagnetic radiation (photons), including X-rays and gamma rays, which can deliver radiation to a relatively large area, and particulate radiation (also called particle beams), such as electrons, protons, and neutrons, which can penetrate only a short distance into the tissue. Radiation dose to the target tissue depends on a number of factors, including the type and location of cancer. The response of the cells to radiation, in turn, depends, among others, on the type and dose of radiation and the sensitivity of the tissue. Ideally, the radiations would target the killing of tumor cells with minimal effects on normal cells. Nevertheless, ionizing radiation during radiation therapy affects healthy organs and tissues as well as cancerous tissues.

Radiation treatment is often associated with short-term side effects, including skin erythema, irritation and inflammation, and medium-term and long-term side effects, such as edema, pain, fibrosis and dilated superficial blood vessels (telangiectasia). Radiation therapy for the treatment of the thoracic walls following mastectomy, head and neck tumors and skin tumors may cause acute reactions and severe damage to the skin and mucous membranes. Skin reactions may vary from acute erythema to desquamation and necrosis. Similarly, the mucous membranes in the mouth, throat, esophagus, trachea, bowel, bladder and rectum may be damaged. Soreness and ulceration in the mouth are common symptoms in patients after treatment with ionizing radiation. As the acute effects of radiation are felt in the accessory glands producing saliva or mucous, side effects also include xerostomia (dry mouth), xerophthalmia (dry eyes) and dryness of the vaginal mucosa.

Long-term complications generally occur at higher doses of radiation (over 35 gray). Late side effects that may develop during the course of several months or years include scarring of tissues, due to the increase in connective tissue, secondary cancers, such as breast, stomach, lung and melanoma, that develop in areas of the body adjacent to the radiation area, and thyroid disorders.

Advance or large tumors in deep organs in the body, such as those in the liver, lung, pancreas, ovaries, rectum, prostate, breast and stomach, often require thermotherapy or heating in addition to ionizing radiation. Cancerous tissue is usually destroyed by exposing the deep tissue to temperatures in the range of 43°-50° C., causing burning of the skin.

The cytotoxic effects of radiation therapy are related to an increase in the energy level of electrons that causes the ionization of DNA, and the production of reactive oxygen species (ROS), including superoxide anion radicals, hydrogen peroxide and hydroxyl radicals, which can damage cells, proteins and DNA.

Space travelers are also exposed to penetrating ionizing radiation. Space radiations include proton and high mass (H), high atomic number (Z) and high energy (E) particle (HZE particle) radiations. The damage caused by space radiations occurs at the time of radiation exposure.

The present inventors discovered that topical application of Nrf2 inducers to areas of the skin and mucosa exposed to ionizing radiations and surrounding areas markedly improves the mechanical resilience of skin and mucous membranes and prevents or reduce skin and mucosa damage in mammals, and specifically in humans exposed to radiation therapy, thermotherapy or space radiations. In particular, topical administration of a pharmaceutically effective amount of sulforaphane before, during or after exposure to radiation therapy provides effective protection against short-term and long-term damage to the skin and mucous membranes.

For the purposes of this invention, the term “patient” denotes an animal. In a preferred aspect of the invention, the patient is a mammal. In the most preferred aspect of the invention, the mammal is a human.

A damage to the skin or mucosa or a disorder of the skin or mucosa, as used in the current context, should be obvious to the person skilled in the art, and is meant to include any abnormality in the skin and mucosa, where radiation therapy is involved in the etiology of the damage or disorder. Examples of damages or diseases for which the current invention could be used preferably include, but are not limited to, acute erythema, skin irritation, inflammation, edema, desquamation, necrosis of the skin, soreness and ulceration in the mouth, pain, fibrosis, telangiectasia, xerostomia, xerophthalmia, dryness of the vaginal mucosa, breast cancer, stomach cancer, lung cancer, melanoma and thyroid disorders.

The treatment envisioned by the invention can be used for patients with a pre-existing condition or for patients pre-disposed to a skin or mucous membrane disease. Additionally, the methods of the invention can be used to alleviate symptoms of radiation therapy in patients, or as a preventative measure in patients.

As used herein, “a pharmaceutically effective amount” is intended to mean an amount effective to elicit a cellular response that is clinically significant.

Transcription factor NF-E2-related factor 2 (Nrf2) belongs to the CNC (Cap-N-Collar) family of transcription factors and possesses a highly conserved basic region-leucine zipper (bZip) structure. Nrf2 plays a critical role in the constitutive and inducible expression of anti-oxidant and detoxification genes, commonly known as phase II genes, that encode defensive enzymes, including drug metabolizing enzymes, such as glutathione S-transferase, NADP(H): quinone oxidoreductase and UDP-glucuronosyltransferase, and anti-oxidant enzymes, such as heme oxygenase-1 (HO-1)1 and -glutamylcysteine synthetase (GCS), in response to oxidative and xenobiotic stress (Braun et al., 2002; Fahey et al., 1997; Fahey and Talalay, 1999; Holtzclaw et al., 2004; Motohashi and Yamamoto, 2004). These enzymes are regulated through a promoter called anti-oxidant responsive element (ARE) or electrophile response element (EpRE). Phase II genes are responsible for cellular defense mechanisms that include the scavenging of reactive oxygen or nitrogen species (ROS or RNS), detoxification of electrophiles and maintenance of intracellular reducing potential (e.g., Holtzclaw et al., 2004; Motohashi and Yamamoto, 2004).

Nrf2 is normally sequestered in the cytoplasm of the cells by an actin-bound regulatory protein called Keap1. When cells are exposed to oxidative or electrophilic stress, the Keap1-Nrf2 complex undergoes a conformational change, and Nrf2 is liberated from the complex and released into the nucleus. The active Nrf2 dimerizes with small Maf proteins, binds to ARE and activates phase II gene transcription (Braun et al., 2002; Motohashi and Yamamoto, 2004).

There is increasing evidence that the induction of phase II enzymes protects from carcinogenesis and mutagenesis and enhances the antioxidant capability of the cells (Fahey and Talalay, 1999; Iida et al., 2004). To date, nine classes of phase II enzyme inducers have been identified: 1) diphenols, phenylene diamines and quinones; 2) Michael acceptors; 3) isothiocyanates; 4) hydroperoxides and hydrogen peroxide; 5) 1,2-dithiole-3-thiones; 6) dimercaptans; 7) trivalent arsenicals; 8) divalent heavy metals; and 9) carotenoids, curcumin and related polyenes (Fahey and Talalay, 1999). These phase II enzyme inducers are considered very efficient antioxidants because unlike direct antioxidants, they are not consumed stoichiometrically during oxido-reduction reactions, have long duration of action, support the function of direct antioxidants, such as tocopherols and CoQ, and enhance the synthesis of glutathione, a strong antioxidant (Fahey and Talalay, 1999).

The diuretic ethacrynic acid (EA), an electrophilic Michael acceptor, oltipraz, and the isothiocyanate sulforaphane have been shown to inhibit lipopolysaccharide (LPS)-induced secretion of high-mobility group box 1 (HMGB1), a proinflammatory protein implicated in the pathogenesis of inflammatory diseases, from immunostimulated macrophages (Killeen et al., 2006). Oltipraz prevents carcinogenesis in liver and urinary bladder by enhancing carcinogen detoxification (Iida et al., 2004). The cytoprotective effect of keratinocyte growth factor (KGF) against oxidative stress in injured and inflamed tissues, including wounded skin, has been related to KGF's stimulation of Nrf2 during cutaneous wound repair (Braun et al., 2002).

Isothiocyanates, which are primarily derived from in cruciferous vegetables, are potent antioxidants and effective agents in the chemoprevention of tumors via the activation of phase II enzymes, inhibition of carcinogen-activating phase I enzymes and induction of apoptosis (Hecht, 1995; Zhang and Talalay, 1994; Zhang et al, 1994). Isothiocyanates are formed in plants from the hydrolysis of glucosinolates, which are β-thioglucoside-N-hydroxysulfates, when maceration of the vegetables by predators, food preparation or chewing causes disruption of the cells with consequent activation and release of the enzyme myrosinase. The resultant aglycones undergo non-enzymatic intramolecular rearrangement to yield isothiocyanates, nitriles and epithionitriles.

Sulforaphane is the aglycone breakdown product of the glucosinolate glucoraphanin, also known as sulforaphane glucosinolate (SGS). The molecular formula of sulforaphane is C₆H₁₁NOS₂, and its molecular weight is 177.29 daltons. Sulforaphane is also known as 4-methylsulfinylbutyl isothiocyanate and (−)-1-isothiocyanato-4(R)-(methylsulfinyl) butane. The structural formula of sulforaphane is:

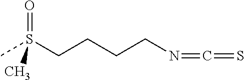

Sulforaphane was recently identified in broccoli and shown to be a potent phase II enzyme inducer in isolated murine hepatoma cells (Zhang et al., 1992), block the formation of mammary tumors in Sprague-Dawley rats (Zhang et al., 1994), prevent promotion of mouse skin tumorigenesis (Gills et al., 2006; Xu et al., 2006) and increase heme oxygenase-1 (HO-1) expression in human hepatoma HepG2 cells (Keum et al., 2006). Sulforaphane was also shown to inhibit ultraviolet (UV) light-induced activation of the activator protein-1 (AP-1), a promoter of skin carcinogenesis, in human keratinocytes (Zhu et al., 2004), and there is evidence that topical application of sulforaphane extract increases the level of phase II enzymes NAD(P)H: quinone oxidoreductase 1 (NQO1), glutathione S-transferase A1 and heme oxygenase 1 in mouse skin epidermis (Dinkova-Kostova et al., 2007). Moreover, sulforaphane protects human epidermal keratinocytes against sulfur mustard, a potent cytotoxic agent and powerful mutagen and carcinogen (Gross et al., 2006), and inhibits cell growth, activates apoptosis, inhibits histone deacetylase (HDAC) activity and decreases the expression of estrogen receptor-α, epidermal growth factor receptor and human epidermal growth factor receptor-2, which are key proteins involved in breast cancer proliferation, in human breast cancer cells (Pledgie-Tracy et al., 2007). Further, sulforaphane was showed to eradicate Helicobacter pylori from human gastric xenografts (Haristoy et al., 2003).

The present invention relates to methods of inducing transcription factor NF-E2-related factor 2 (Nrf2) as a way to prevent or treat damages to the skin or mucosa or disorders of the skin or mucosa caused by radiation therapy, hyperthermia or space radiations as described above.

The compounds used in the methods of the invention are inducers of Nrf2 activity, as described above.

Isothiocyanates are compounds containing the isothiocyanate (NCS) moiety and are easily identifiable by one of ordinary skill in the art. An example of an isothiocyanate includes, but is not limited to sulforaphane or its analogs. The description and preparation of isothiocyanate analogs is described in U.S. Reissue Pat. 36,784, and is hereby incorporated by reference in its entirety. The sulforaphane analogs used in the present invention include 6-isothiocyanato-2-hexanone, exo-2-acetyl-6-isothiocyanatonorbornane, exo-2-isothiocyanato-6-methylsulfonylnorbornane, 6-isothiocyanato-2-hexanol, 1-isothiocyanato-4-dimethylphosphonylbutane, exo-2-(1′-hydroxyethyl)-5-isothiocyanatonorbomane, exo-2-acetyl-5-isothiocyanatonorbornane, 1-isothiocyanato-5-methylsulfonylpentane, cis-3-(methylsulfonyl)cyclohexylmethylisothiocyanate and trans-3-(methylsulfonyl)cyclohexylmethylisothiocyanate.

Glucosinolates, the precursors to isothiocyanates, are also contemplated by the present invention. Glucosinolates are well-known in the art and are reviewed in Fahey et al., Phytochemistry, 56:5-51 (2001), the entire contents of which are hereby incorporated by reference.

Other compounds contemplated by the present invention include keratinocyte growth factor (KGF), oltipraz, ethacrynic acid, and analogs thereof, as well a additional Michael reaction acceptors, such as triterpenoids or cyclic/acyclic bis-benzylidene-alkalones.

The compounds used in the methods of the present invention can be formulated into pharmaceutical compositions with suitable, pharmaceutically acceptable excipients for topical administration to mammals. Such excipients are well known in the art. Topical administration includes administration to the skin or mucosa, including surfaces of the lung, stomach, vagina, mouth and eye.

Dosage forms for topical administration include, but are not limited to, ointments, creams, emulsions, lotions, gels, sunscreens and agents that favor penetration within the epidermis. In a preferred embodiment, the composition is in the form of topical ointment.

Various additives, known to those skilled in the art, may be included in the topical formulations of the invention. Examples of additives include, but are not limited to, solubilizers, skin permeation enhancers, preservatives (e.g., anti-oxidants), moisturizers, gelling agents, buffering agents, surfactants, emulsifiers, emollients, thickening agents, stabilizers, humectants, dispersing agents and pharmaceutical carriers.

Examples of moisturizers include jojoba oil and evening primrose oil.

Suitable skin permeation enhancers are well known in the art and include lower alkanols, such as methanol ethanol and 2-propanol; alkyl methyl sulfoxides such as dimethylsulfoxide (DMSO), decylmethylsulfoxide (C₁₀ MSO) and tetradecylmethyl sulfoxide; pyrrolidones, urea; N,N-diethyl-m-toluamide; C₂-C₆ alkanediols; dimethyl formamide (DMF), N,N-dimethylacetamide (DMA) and tetrahydrofurfuryl alcohol.

Examples of solubilizers include, but are not limited to, hydrophilic ethers such as diethylene glycol monoethyl ether (ethoxydiglycol, available commercially as Transcutol®) and diethylene glycol monoethyl ether oleate (available commercially as Softcutol®); polyoxy 35 castor oil, polyoxy 40 hydrogenated castor oil, polyethylene glycol (PEG), particularly low molecular weight PEGs, such as PEG 300 and PEG 400, and polyethylene glycol derivatives such as PEG-8 caprylic/capric glycerides (available commercially as Labrasol®); alkyl methyl sulfoxides, such as DMSO; pyrrolidones, DMA, and mixtures thereof.

Suitable pharmaceutical carriers include any such materials known in the art, e.g., any liquid, gel, solvent, liquid diluent, solubilizer, polymer or the like, which is nontoxic and which does not significantly interact with other components of the composition or the skin in a deleterious manner.

Prevention and/or treatment of infections can be achieved by the inclusion of antibiotics, as well as various antibacterial and antifungal agents, for example, paraben, chlorobutanol, phenol sorbic acid, and the like, in the compositions of the invention.

One of ordinary skill will appreciate that effective amounts of the agents in the compositions used in the methods of the invention can be determined empirically. It will be understood that, when administered to a human patient, the total daily usage of the composition of the present invention will be decided by the attending physician within the scope of sound medical judgment. The specific therapeutically effective dose level for any particular patient will depend upon a variety of factors: the type and degree of the response to be achieved; the activity of the specific composition employed; the age, body weight, general health, sex and diet of the patient; the duration of the treatment; drugs used in combination or coincidental with the method of the invention; and like factors well known in the medical arts.

Typically, the amount of Nrf2 inducer in the composition topically administered to the patient will be from about 100 nmol to about 1 μmol/cm², and the composition will be applied directly on the skin over relevant portions of the body of the patient so as to prevent or minimize short-term and long-term side effects resulting from radiation therapy or hyperthermia.

The potential commercial uses of the disclosed preparations include, for example, (i) protective/prophylactic, (ii) cosmetic and (iii) medical uses. In one embodiment, protective lotions and cremes for topical application either oil-(sulforaphane) or water-based (glucoraphanin plus hydrolyzing agent) are provided. In another embodiment, sulforaphane-containing compositions can be combined with sunscreens.

Compositions comprising the Nrf2 inducers described above can also be administered in a variety of other routes, including oral, mucosal, subcutaneous, intramuscular and parenteral administration, and may comprise a variety of carriers or excipients. Suitable carrier may include, but are not limited to, a non-toxic solid, semisolid or liquid filler, diluent, encapsulating material or formulation auxiliary of any type, such as liposomes.

Compositions for parenteral injection can comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants. The compositions of the present invention can also contain adjuvants such as, but not limited to, preservatives, wetting agents, emulsifying agents, and dispersing agents. It can also be desirable to include isotonic agents such as sugars, sodium chloride, and the like. Prolonged absorption of the injectable pharmaceutical form can be brought about by the inclusion of agents which delay absorption such as aluminum monostearate and gelatin.

In some cases, to prolong the effect of the drugs, it is desirable to slow the absorption from subcutaneous or intramuscular injection. This can be accomplished by the use of a liquid suspension of crystalline or amorphous material with poor water solubility. The rate of absorption of the drug then depends upon its rate of dissolution which, in turn, can depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues. The injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use.

Solid dosage forms for oral administration include, but are not limited to, capsules, tablets, pills, powders, and granules. In such solid dosage forms, the active compounds are mixed with at least one item pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form can also comprise buffering agents. Solid compositions of a similar type can also be employed as fillers in soft and hard filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings, such as extended-release, sustained-release, delayed release and immediate-release coatings well known in the pharmaceutical formulating art. They can optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. The active compounds can also be in micro-encapsulated form, if appropriate, with one or more of the above-mentioned excipients.

Liquid dosage forms for oral administration include, but are not limited to, pharmaceutically acceptable emulsions, solutions, suspensions, syrups and elixirs. In addition to the active compounds, the liquid dosage forms can contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents. Suspensions, in addition to the active compounds, can contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

A dietary composition according to the present invention is any ingestible preparation containing sulforaphane, isothiocyanates, glucosinolates or analogs thereof. For example, sulforaphane, isothiocyanates, glucosinolates or analogs thereof may be mixed with a food product. The food product can be dried, cooked, boiled, lyophilized or baked. Breads, teas, soups, cereals, salads, sandwiches, sprouts, vegetables, animal feed, pills, and tablets, are among the vast number of different food products contemplated in the present invention.

### EXAMPLES

### Example 1

### Preparation of Sulforaphane from Broccoli Sprouts

Seeds of broccoli (Brassica oleracea italica, cv. DeCicco), certified not to have been treated with any pesticides or other seed treatment chemicals, were sprouted and processed as described by Fahey et al. (12). Briefly, seeds were surface-disinfected with a 25% aqueous solution of Clorox® bleach containing a trace of Alconox® detergent and exhaustively rinsed with water. The seeds were then spread out in a layer in inclined, perforated plastic trays, misted with filtered water for 30 s about 6 times/h and illuminated from overhead fluorescent lamps. Growth was stopped after 3 days by plunging sprouts directly into boiling water in a steam-jacketed kettle, returning to a boil, and stirring for ˜5 min. This treatment inactivated the endogenous sprout myrosinase and extracted the glucosinolates. Glucoraphanin, the precursor of sulforaphane, was the predominant glucosinolate in the initial extract as determined by HPLC (26). Daikon sprout myrosinase was then added for quantitative conversion of glucosinolates to isothiocyanates as described by Fahey et al., 1997 and Shapiro et al., 2001 (12,27). This preparation was then lyophilized, dissolved in ethyl acetate, evaporated to dryness by rotary evaporation, dissolved in a small volume of water, and acetone was added to a final concentration of 50 mM sulforaphane in 80% acetone:20% water (v/v). The total isothiocyanate content was determined (12,27) by the cyclocondensation reaction (28), complete absence of glucosinolates was confirmed by HPLC (26), and the precise ratio of the isothiocyanates liberated by the myrosinase reaction was determined by HPLC on an acetonitrile gradient, and matched the glucosinolate profile of the extract. Sulforaphane constituted more than 90% of the isothiocyanate content. This preparation was diluted in 80% acetone (v/v) to produce the “high dose” (1.0 μmol/100 μl) and “low dose” (0.3 μmol/100 μl). Bioassay in the Prochaska test (29,30) yielded a CD value (concentration required to double the activity of NQO1) consistent with previous experiments (11).

### Example 2

### Treatment of Keratinocytes with Sulforaphane

Glutathione is the primary and most abundant cellular nonprotein thiol and constitutes a critical part of the cellular defense: it reacts readily with potentially damaging electrophiles and participates in the detoxification of reactive oxygen intermediates and their toxic metabolites by scavenging free radicals and reducing peroxides. The capacity to increase cellular levels of GSH is critically important in combating oxidative stress. To this end, we examined the ability of the sulforaphane-induced phase 2 response to protect against oxidative stress caused by UVA in cultures of keratinocytes. We chose UVA for this study, because its genotoxicity is thought to be primarily due to the generation of reactive oxygen intermediates.

Cell Cultures

HaCaT human keratinocytes (a gift from G. Tim Bowden, Arizona Cancer Center, Tucson) were cultured in Dulbecco's modified Eagle's medium (DMEM) supplemented with 5% FBS; and PE murine keratinocytes (a gift from Stuart H. Yuspa, National Cancer Institute, Bethesda, Md.) were cultured in Eagle's minimum essential medium (EMEM) with 8% FBS, treated with Chelex resin (Bio-Rad) to remove Ca²⁺.

Quinone Reductase (NQO1) and Glutathione Assays

Cells (20,000 per well) were grown for 24 h in 96-well plates, then exposed to serial dilutions of sulforaphane for either 24 h (for glutathione determination) or 48 h (for NQO1 determination), and finally lysed in 0.08% digitonin. An aliquot (25 μl) was used for protein analysis. Activity of NQO1 was determined by the Prochaska test (29,30). To measure the intracellular glutathione levels, 25 μl of cell lysate received 50 μl of ice-cold metaphosphoric acid (50 g/liter) in 2 mM EDTA to precipitate cellular protein. After 10 min at 4° C., plates were centrifuged at 1,500 g for 15 min and 50 μl of the resulting supernatant fractions were transferred to a parallel plate. To each of these wells, 50 μl of 200 mM sodium phosphate buffer, pH 7.5, containing 10 mM EDTA, were added and total cellular glutathione was determined by rate measurements in a recycling assay (31,32).

UV Irradiation of Cells and Determination of Reactive Oxygen Intermediates

PE cells (50,000 per well) were seeded into 24-well plates and grown for 48 h. The cells were then exposed to 1 μM or 5 μM sulforaphane for 24 h. On the day of the experiments, after removing the medium, the cells were incubated with 100 μM 2′,7′-dichlorodinitrofluorescein diacetate in 500 μl of fresh medium (Molecular Probes, Eugene, Oreg.) for 30 min. The medium containing the fluorescent probe was then removed, the cells were washed with DPBS, and exposed to UVA radiation (10 J/cm²). Control cells were kept in the dark. Cells were detached with trypsin, suspended in 2.0 ml of DPBS, and the intensity of fluorescence was determined in cell suspensions at 520 nm with an excitation of 485 nm in 2-ml cuvettes in a Perkin-Elmer LS50 spectrofluorimeter.

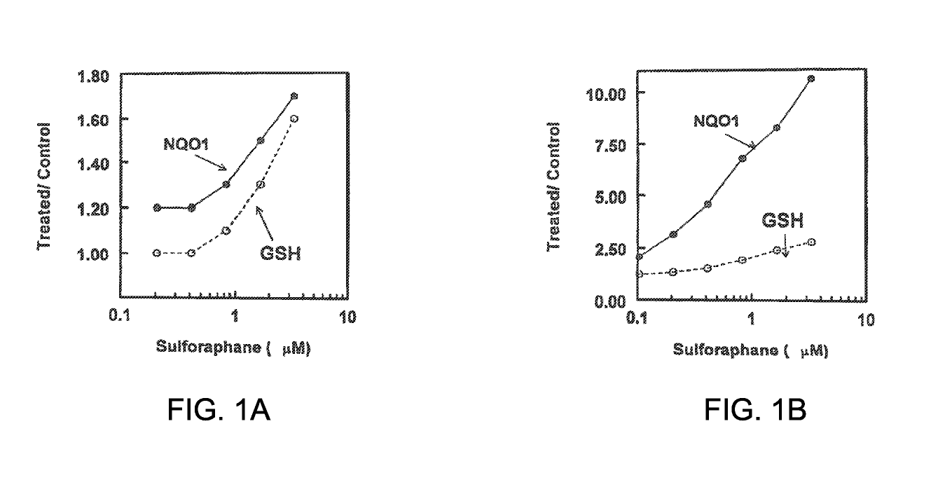

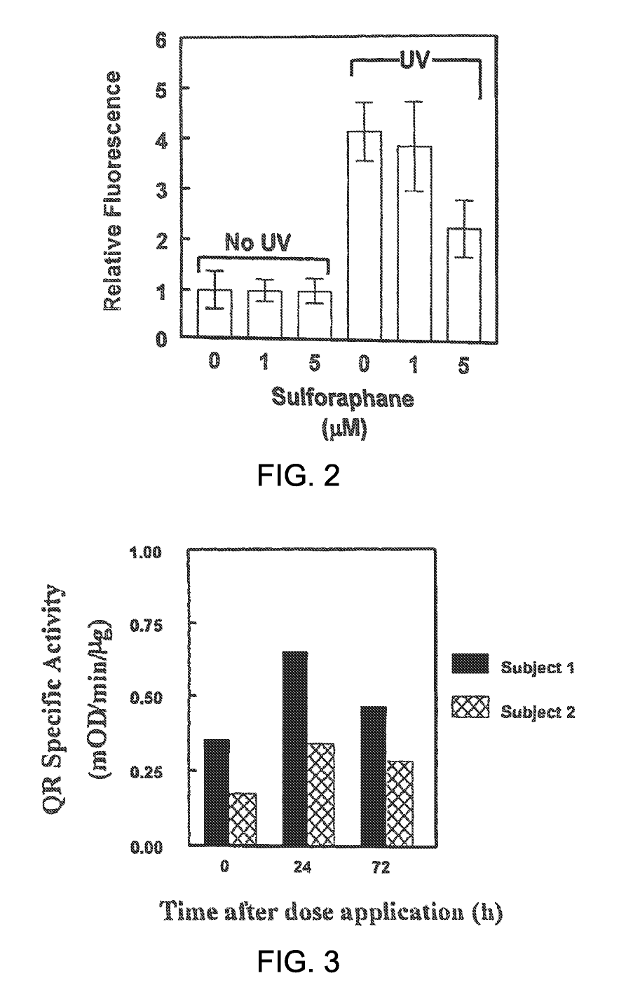

When HaCaT human keratinocytes or PE murine keratinocytes were exposed to sulforaphane, the intracellular levels of NQO1 and glutathione were increased in a dose-dependent manner (FIG. 1A, B) in agreement with previous observations (Ye and Zhang, 2001). Especially striking was the magnitude of NQO1 induction (>10-fold) in HaCaT cells without any apparent evidence of cytotoxicity. Treatment with 5 μM sulforaphane for 24 h produced a substantial (50%) reduction in reactive oxygen intermediates generated by the UV radiation as quantified by the fluorescent probe 2′,7′-dichlorodinitro-fluorescein (35) (FIG. 2).

### Example 3

### Effect of Topical Application of Sulforaphane on NQO1 and GSH in Mice

The phase 2 response was next evaluated in vivo in SIGH-1 hairless mice. Female SKH-1 hairless mice (4 weeks old) were obtained from Charles River Breeding Laboratories (Wilmington, Mass.) and were acclimatized in our animal facility for 2 weeks before the start of the experiment. The animals were kept on a 12-h light/12-h dark cycle, 35% humidity, and given free access to water and pelleted AIN 76A diet (Harlan TekLad, free of inducers). All animal experiments were in compliance with the National Institutes of Health Guidelines and were approved by the Johns Hopkins University Animal Care and Use Committee.

Seven-week-old SKH-1 hairless mice (5 per group) were treated topically on their backs with either 100 μl of a standardized myrosinase-hydrolyzed broccoli sprout extract containing 1 μmol of sulforaphane, or vehicle (100 μl of 80% acetone:20% water, v/v). The animals were euthanized 24 h later and their dorsal skins were dissected using a rectangular template (2.5×5 cm) and frozen in liquid N₂. Skin samples were pulverized in liquid N₂ and 100 mg of the resulting powder was homogenized in 1 ml of either 0.25 M sucrose buffered with 10 mM Tris-HCl, pH 7.4, for analysis of NQO1 enzymatic activity and protein content, or ice-cold metaphosphoric acid (50 g/liter) in 2 mM EDTA for analysis of glutathione. Centrifugation at 14,000 g for 20 min at 4° C. yielded clear supernatant fractions, aliquots of which were used for determination of protein content, enzyme activity, and total glutathione levels as described below for the cell culture experiments.

The results showed that topical administration of sulfopharane produced about a 50% induction of NQO1 (P<0.001) and about a 15% elevation of the total glutathione levels of the treated animals compared to the controls.

### Example 4

### Effect of Topical Application of Sulforaphane on NQO1 and GSH in Humans

This study involving healthy human volunteers was done in accordance with protocols approved by the Institutional Review Board at the Johns Hopkins University. The safety of topical administration of single doses of broccoli sprout extracts to the skin of healthy human volunteers was studied. The extracts were prepared in 80% acetone:20% water and their sulforaphane content was precisely determined by cyclocondensation assay, a method routinely used in our laboratory for quantification of isothiocyanates and their dithiocarbamate metabolites. A circle (1 cm in diameter) was drawn on the skin of volar forearm of each participant and the extract was then applied inside the circle by using a positive displacement pipette. Two subjects participated for each of the 8 escalating doses that were administered (0.3; 5.3; 10.7; 21.4; 42.7; 85.4; 170; and 340 nmol of sulforaphane). Each subject served as his/her own control and received a placebo “vehicle spot.” No adverse reactions were observed at any of these doses.

Efficacy studies were also performed. The endpoint was determination of the enzyme activity of quinone reductase (a prototypic Phase 2 protein) in 3-mm skin punch biopsies of 2 healthy human volunteers after application of a single dose of broccoli sprout extract. Again, each subject served as his/her own control and received a “vehicle spot”. Both quinone reductase activity and protein content were reliably detected in these samples. The specific activity of quinone reductase was increased by ˜2-fold 24 h after application of an extract containing 100 nmol of sulforaphane (FIG. 3). Notably, the induction was long-lasting as the activity remained higher than that of the placebo-treated sites even when the biopsies were performed 72 h after application.

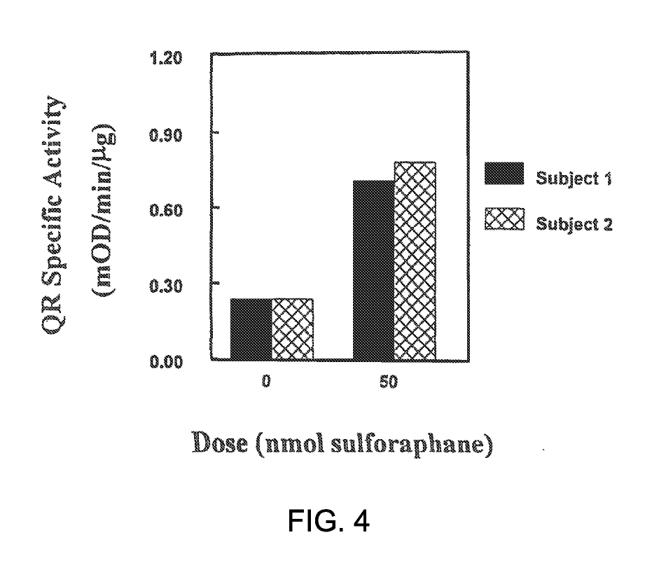

The effect of three repeated topical applications (at 24-h intervals) of broccoli sprout extract containing 50 nmol of sulforaphane was studied next. This led to even greater elevations of quinone reductase (NQO1) specific activity in the underlying skin of two healthy human volunteers (FIG. 4).

### Example 5

### Effect of Sulforaphane on Inducible Nitric Oxide Synthase

We have recently found a linear correlation spanning over 6 orders of magnitude of potencies between inhibition of inflammatory responses (iNOS and COX-2 activation by γ-interferon) and induction of phase 2 enzymes among a series of synthetic triterpenoids (20).

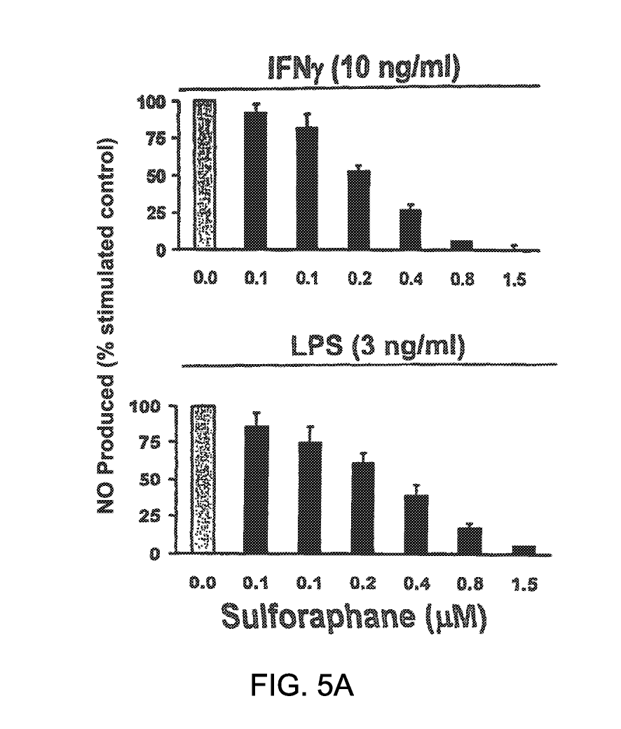

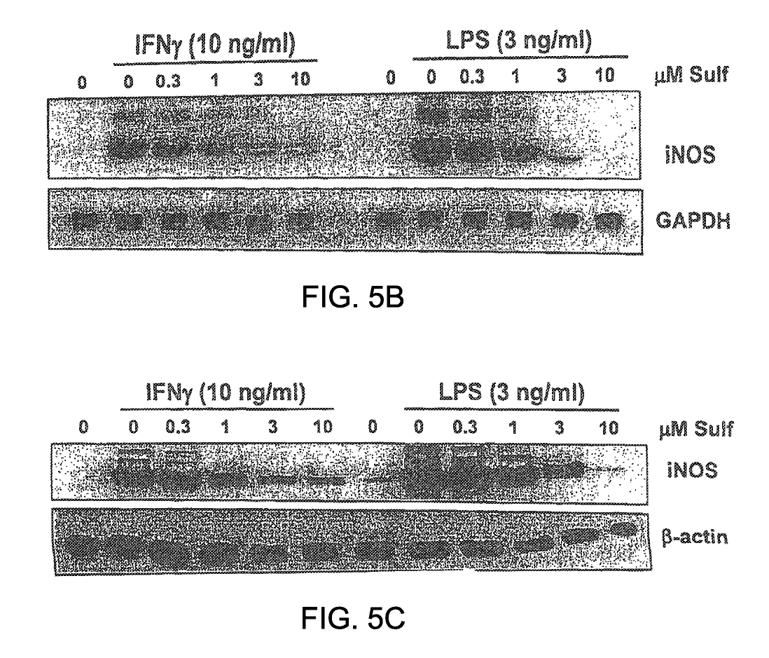

RAW 264.7 macrophages (5×10⁵ cells/well) were plated in 96-well plates and incubated with sulforaphane and either 10 ng/ml of IFN-γ or 3 ng/ml of LPS for 24 h. NO was measured as nitrite by the Griess reaction (33). When RAW 264.7 cells were incubated with γ-interferon or lipopolysaccharide together with various concentrations of sulforaphane for 24 h, there was a dose-dependent inhibition of NO formation with an IC₅₀ of 0.3 μM for both cytokines (FIG. 5A).

In agreement with this result, Northern and Western blot analyses revealed that the synthesis of iNOS mRNA and protein were also inhibited (FIG. 5B, C). RAW 264.7 macrophages (2×10⁶ cells/well) were incubated with sulforaphane and either 10 ng/ml of IFN-γ or 3 ng/ml of LPS overnight. For Northern blots, total RNA was isolated with Trizol reagent (Invitrogen) and prepared for blotting as previously described (33). Probes for iNOS and GAPDH were radiolabeled with [γ³²P]dCTP with random primers. For Western blots, total cell lysates were subjected to SDS/PAGE, transferred to a membrane, and probed with iNOS and β-actin antibodies (Santa Cruz Biotechnology).

These findings indicate that exposure to sulforaphane suppresses induction of iNOS by either γ-interferon or lipopolysaccharide and attenuates inflammatory responses that play a role in the process of carcinogenesis.

### Example 6

### Effect of Topical Application of Sulforaphane on UV Light-Induced Carcinogenesis

Exposure of SKH-1 hairless mice to relatively low doses of UVB radiation (30 mJ/cm²) twice a week for 20 weeks results in “high-risk mice” that subsequently develop skin tumors in the absence of further UV treatment (24,25). This animal model is highly relevant to humans who have been heavily exposed to sunlight as children, but have limited their exposure as adults. In addition, it allows the evaluation of potential chemoprotective agents after completion of the irradiation schedule, thus excluding the possibility of a “light filtering effect” by the protective preparations of sprout extracts that may be slightly colored. Thus, UVB-pretreated high-risk mice were treated topically once a day 5 days a week for 11 weeks with 100 μl of standardized myrosinase-hydrolyzed broccoli sprout extracts containing either 0.3 μmol (low dose) or 1 μmol (high dose) of sulforaphane. The control group received vehicle treatment. Body weights and formation of tumors larger than 1 mm in diameter were determined weekly.

UVB radiation was provided by a bank of UV lamps (FS72T12-UVB-HO, National Biological Corporation, Twinsburg, Ohio) emitting UVB (280-320 nm, 65% of total energy) and UVA (320-375 nm, 35% of total energy). The radiant dose of UVB was quantified with a UVB Daavlin Flex Control Integrating Dosimeter and further calibrated with an IL-1400 radiometer (International Light, Newburyport, Mass.).

The animals were irradiated for 20 weeks on Tuesdays and Fridays with a radiant exposure of 30 mJ/cm²/session. One week later, the mice were divided into three groups: 29 animals in each treatment group and 33 animals in the control group. The mice in the two treatment groups received topical applications of either 100 μl of broccoli sprout extract containing 1 μmol sulforaphane (high dose), or 0.3 μmol of sulforaphane (low dose), those in the control group received 100 μl of vehicle. Treatment was repeated 5 days a week for 11 weeks at which time all animals in the control group had at least one tumor and the experiment was ended. Tumors (defined as lesions >1 mm in diameter) and body weight were recorded weekly. Tumor volumes were determined by measuring the height, length, and width of each mass that was larger than 1 mm in diameter. The average of the three measurements was used as the diameter and the volume was calculated (v=4πr³/3). All mice were euthanized on the same day and the size and multiplicity of tumors was determined. Dorsal skins were dissected using a rectangular template (2.5×5 cm) to include the entire treated areas of the mice. Skins were stapled to cardboard, photographed, and fixed in ice-cold 10% phosphate-buffered formalin at 4° C. for 24 h.

There was no difference in average body weight and weight gain among the groups. The body weights (mean±SD) at the onset of the experiment were: 22.3±1.9 g for the control group, 22.2±1.9 g for the low-dose-treated, and 23.0±1.9 g for the high dose-treated group. At the end of the experiment (31 weeks later), the respective body weights were: 32.1±9.7 g, 31.9±8.8 g, and 32.1±6.9 g. The earliest lesions larger than 1 mm were observed 2 weeks after the end of irradiation which was 1 week after topical treatment with protector was started. At this time point, 3, 6, and 4 mice of the control, low dose-treated, and high dose-treated mice, respectively, developed their first tumor.

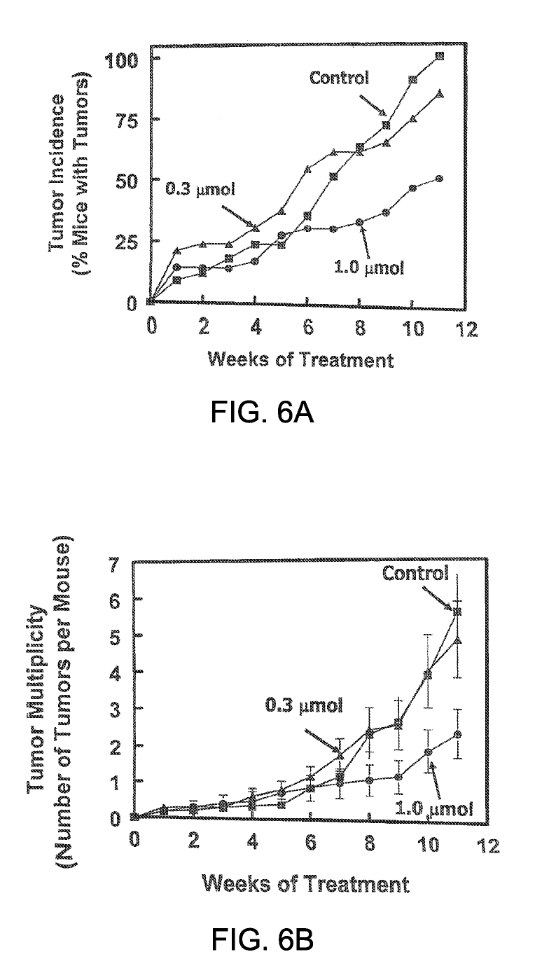

The high dose-treated animals were substantially protected against the carcinogenic effects of UV radiation. Thus, after 11 weeks of treatment when the experiment was terminated, 100% of the animals in the control group had developed tumors, while 48% of the mice treated daily with sprout extract containing 1 μmol of sulforaphane were tumor-free (FIG. 6A). Of note, three animals (two of the control and one of the low-dose-treated groups) were euthanized 1 week before the end of the experiment because they had tumors approaching 2 cm in diameter. Kaplan-Meier survival analysis followed by both a stratified log-rank test, and a Wilcoxon test for equality of survivor functions showed that there was a highly significant difference (P<0.0001) between treatments. The 1-μmol treatment was different from both the 0.3 μmol and the control treatment, at the 95% confidence level, for each of the last three observation periods (weeks 9, 10, and 11). There was no significant difference between the 0.3 μmol and the control treatment at any time point.

FIG. 6B shows the overall effect of treatment on tumor number was highly significant (p<0.001). ANOVA comparisons of the 1.0-μmol dose level with the control indicated a highly significant overall effect (p<0.001), but differences only became significant after week 9: p<0.0794, p<0.0464 and p<0.0087 for observations made at weeks 9, 10, and 11, respectively. Average values±SE are shown.

In addition to the reduction in tumor incidence and multiplicity, there was a significant delay of tumor appearance. Whereas 50% of the control animals at risk bad tumors at 6.5 weeks after the end of radiation, it took 10.5 weeks for 50% of the high-dose treated animals at risk to develop tumors. Of note, the ability of a protective agent to delay the carcinogenic process is becoming an increasingly appreciated concept in chemoprevention. Similarly, tumor multiplicity was reduced by 58%: the average number of tumors per mouse was 2.4 for the treated and 5.7 for the control group.

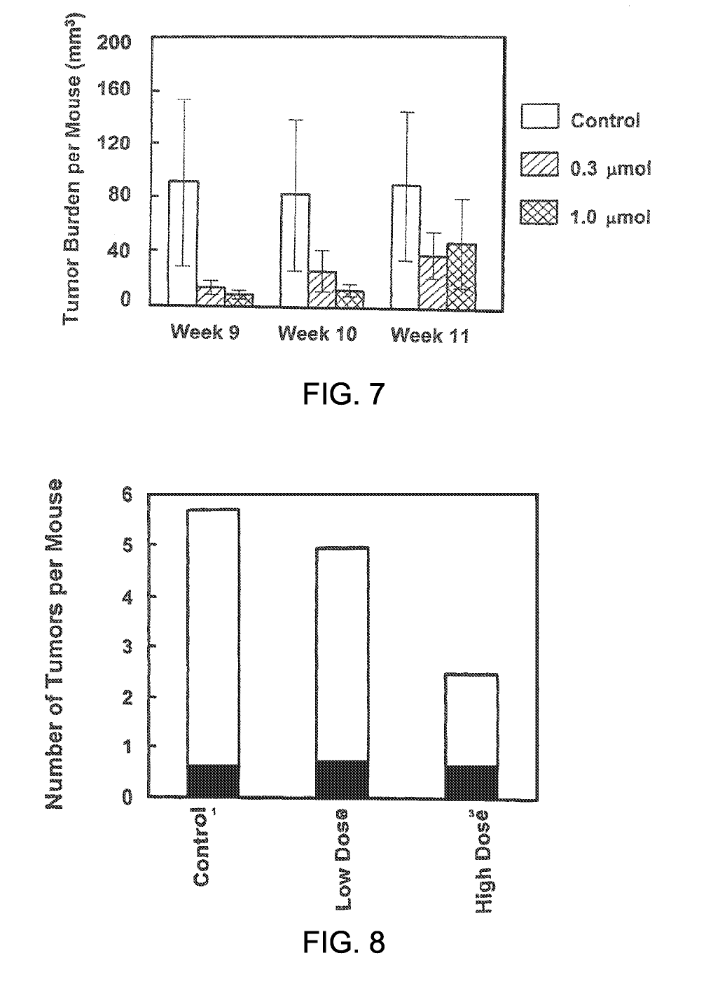

Although there was no difference in tumor incidence and multiplicity between the low-dose-treated and the vehicle-treated groups (FIG. 6A, B), the overall tumor burden (expressed as volume in mm³) per mouse was substantially smaller in the low dose-treated group by 86-, 68-, and 56% at treatment weeks 9, 10, and 11, respectively (FIG. 7). The seemingly decreasing effectiveness with respect to treatment with time appears to occur because the large tumors (>1 cm³) grew rapidly during the last 2 weeks of the experiment. The overall tumor burden in the high dose-treated group was even more dramatically reduced by 91-, 85-, and 46% at treatment weeks 9, 10, and 11, respectively. Interestingly, some of the mice from this treatment group had tumors on the head, where the extract was not applied, but no tumors on their back, where the protective extract was applied.

Although histological characterization of the individual tumors has not been completed, this animal model consistently results in the formation of approximately 80% small nonmalignant tumors (primarily keratoacanthomas and a few papillomas) and approximately 20% large malignant tumors (squamous cell carcinoma) (24,25). We classified all tumors according to their volumes in two categories: “small” (<1 cm³) (FIG. 8, white bars) and “large” (>1 cm³) (FIG. 8, black bars). Treatment with the sprout extract did not change the multiplicity of large tumors across the experimental groups, there were 17 large tumors among all 33 animals in the control group, 19 among all 29 animals in the low dose-treated group, and 16 among all 29 animals in the high dose-treated group. In contrast, the broccoli sprout extract produced a dose-dependent inhibition on the number of small tumors: 170, 123, and 54 in the control, low dose-treated, and high dose-treated groups, respectively. It is possible that the unaffected tumors originated from cells that had accumulated mutations caused by direct UV-radiation-induced DNA photoproducts, whereas the extracts inhibited mainly carcinogenic processes resulting from oxidative stress-induced DNA damage. A similar phenomenon has been reported in that the soybean isoflavone genistein inhibited the generation of lipid peroxidation products, H₂O₂, and 8-hydroxy-2′-deoxyguanosine in mouse skin, but had no effect on the pyrimidine dimers formed in response to UV radiation (36).

### Statistical Analysis

Tumor incidence was evaluated using the Kaplan-Meier survival analysis followed by both a stratified log-rank test and a Wilcoxon test, for equality of survivor functions. Tumor multiplicity was evaluated by ANOVA and comparisons were made on all treatments and on individual, paired treatments (t-test). Tumor volume was evaluated by ANOVA with treatment time as a nested variable. These calculations were performed using Stata 7.0 (College Station, Tex.). Other statistics were calculated using Excel.

### Example 7

### Preparation of Freeze-Dried Broccoli Sprout Extract Powder

Seeds of broccoli (Brassica oleracea italica, cv. DeCicco) were used to grow sprouts as described in Example 1. Growth was arrested after 3 days by plunging sprouts into boiling water and allowed to boil for ˜30 min. This treatment inactivated the endogenous sprout myrosinase and extracted the glucosinolates. Glucoraphanin, the precursor of sulforaphane, was the predominant glucosinolate in the extract as determined by HPLC (26). This preparation was then lyophilized to give glucosinolate-rich powder that contained ˜8.8% of glucoraphanin by weight. The powder was mixed with the mouse diet (powdered AIN 76A) to give the equivalent of 10 μmol (low dose) or 50 μmol (high dose) of glucoraphanin per 3 grams of diet.

### Example 8

### Effect of Dietary Administration of Sulforaphane on UV Light-Induced Carcinogenesis

In this study, UVB-pretreated high-risk mice were fed for 13 weeks a diet into which was incorporated a freeze-dried broccoli sprout extract powder prepared according to Example 6 (equivalent to 10 μmol/day [low dose] and 50 μmol/day [high dose] glucoraphanin, the glucosinolate precursor of sulforaphane that is found in the intact plant, about 10% of which is converted to sulforaphane upon ingestion by mice). The diet of the control group did not contain any freeze-dried broccoli sprout extract powder. Body weights and formation of tumors larger than 1 mm in diameter were determined weekly.

UVB radiation was provided by a bank of UV lamps (FS72T12-UVB-HO, National Biological Corporation, Twinsburg, Ohio) emitting UVB (280-320 nm, 65% of total energy) and UVA (320-375 nm, 35% of total energy). The radiant dose of UVB was quantified with a UVB Daavlin Flex Control Integrating Dosimeter and further calibrated with an IL-1400 radiometer (International Light, Newburyport, Mass.).

The animals were irradiated for 20 weeks on Tuesdays and Fridays with a radiant exposure of 30 mJ/cm²/session. One week later, the mice were divided into three groups: 30 animals in each treatment group and 30 animals in the control group. The mice in the two treatment groups received a diet into which was incorporated a freeze-dried broccoli sprout extract powder. The diet of the low dose treatment group included a freeze-dried broccoli sprout extract powder equivalent to 10 μmol/day glucoraphanin, while the diet of the high dose treatment group included a freeze-dried broccoli sprout extract powder equivalent to 50 μmol/day glucoraphanin. The diet of the control group did not contain a freeze-dried broccoli sprout extract powder. The mice were fed this diet for 13 weeks. After 13 weeks, 93% of the control mice had tumors and the experiment was ended.

Tumor volumes were determined by measuring the height, length, and width of each mass that was larger than 1 mm in diameter. The average of the three measurements was used as the diameter and the volume was calculated (v=4πr³/3). All mice were euthanized on the same day and the size and multiplicity of tumors was determined. Dorsal skins were dissected using a rectangular template (2.5×5 cm) to include the entire treated areas of the mice. Skins were stapled to cardboard, photographed, and fixed in ice-cold 10% phosphate-buffered formalin at 4° C. for 24 h.

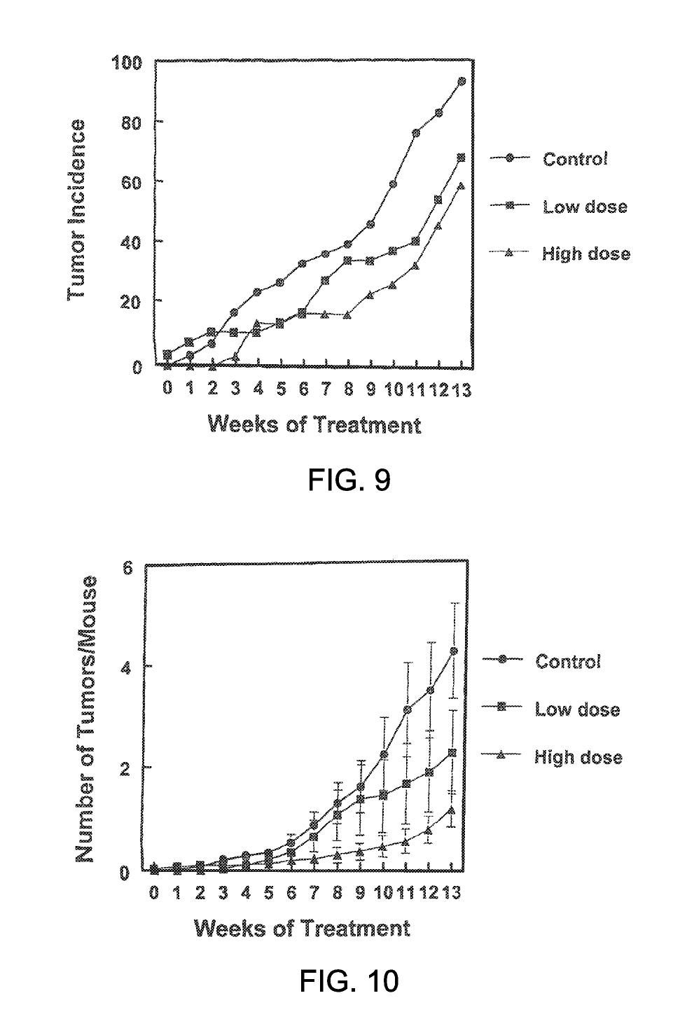

Tumor incidence (percent animals with tumors) was reduced by 25% and 35%, in the animals receiving the low dose and the high dose of glucoraphanin, respectively, as compared to the control group of mice. (FIG. 9)

Even greater was the effect of treatment on tumor multiplicity (number of tumors per mouse) that was reduced by 47% and 72% in the animals receiving the low dose and the high dose of glucoraphanin, respectively, as compared to the control group of mice. Thus, while the animals in the control group had on the average of 4.3 tumors per mouse, the number of tumors per mouse was 2.3 for the low dose and 1.2 for the high dose of glucoraphanin. (FIG. 10)

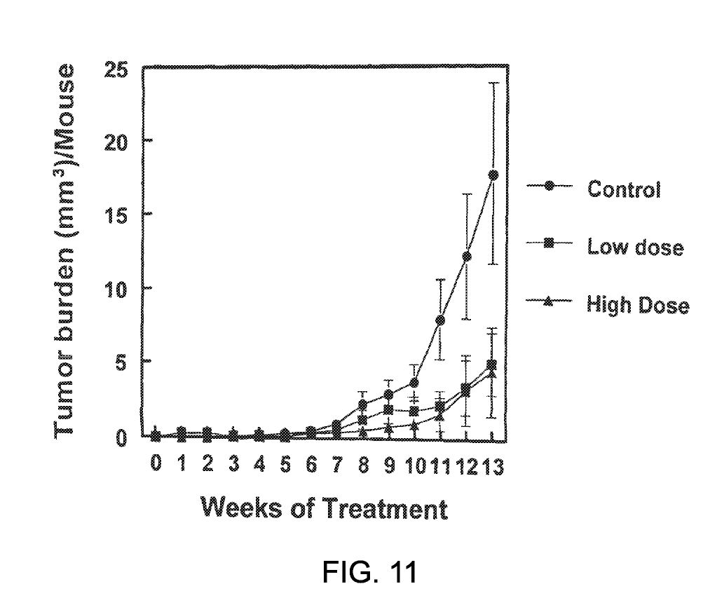

Tumor burden was also affected dramatically: both low dose and high dose of glucoraphanin treatments resulted in 70% inhibition in the total tumor volume per mouse. (FIG. 11)

The plasma levels of sulforaphane and its metabolites were very similar: 2.2 μM and 2.5 μM for the low dose and the high dose of glucoraphanin treatments, respectively, indicating that glucoraphanin was converted to sulforaphane and that the chronic dietary treatment had resulted in steady-state levels of sulforaphane and its metabolites in the blood of the animals. These levels are adequate to expect biological effects.

The levels of phase 2 enzymes were induced (2 to 2.5-fold for quinone reductase 1 and 1.2 to 2.2-fold for glutathione S-transferases) in nearly all the organs that were examined, namely forstomach, stomach, bladder, liver, and retina.

Statistical Analysis

Tumor incidence was evaluated using the Kaplan-Meier survival analysis followed by both a stratified log-rank test and a Wilcoxon test, for equality of survivor functions. Tumor multiplicity was evaluated by ANOVA and comparisons were made on all treatments and on individual, paired treatments (t-test). Tumor volume was evaluated by ANOVA with treatment time as a nested variable. These calculations were performed using Stata 7.0 (College Station, Tex.). Other statistics were calculated using Excel.

### Example 9

### Effect of Topical Application of Sulforaphane on High Dose UV Light-Induced Carcinogenesis

SKH-1 hairless mice were exposed to single high doses (700-1200 mJ/cm²) of narrow-band 311-nm UVB radiation. These high doses are comparable with those used to determine skin erythema in humans. The mice were radiated in ventilated cabinets equipped with UV lamps. The control group received vehicle treatment.

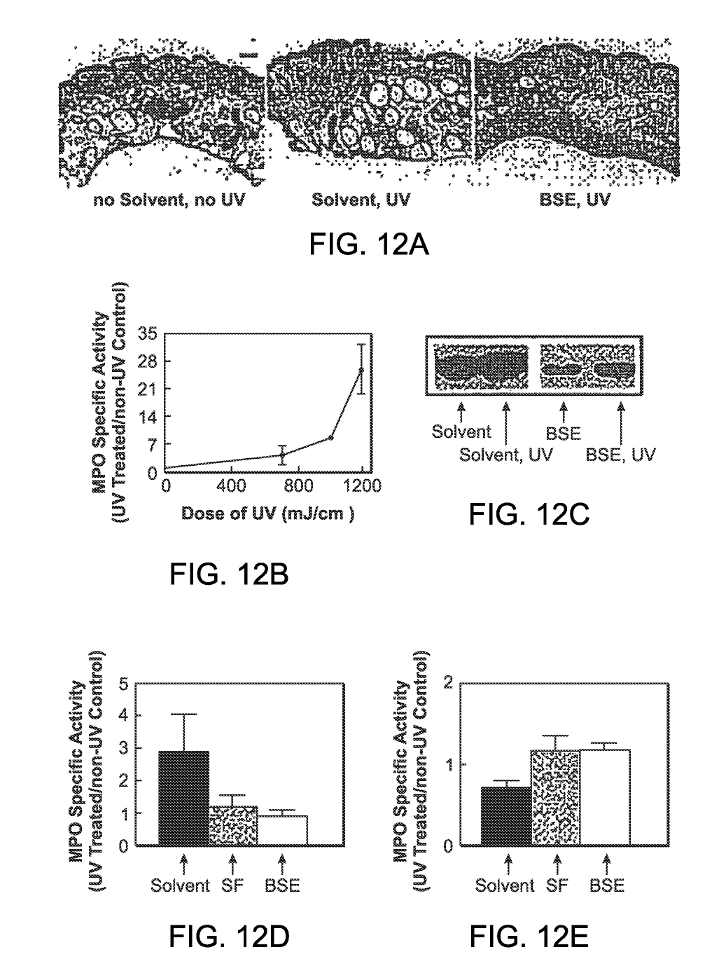

Irradiation caused the skin layers of the mice to became much thicker and showed marked edema and inflammation within 24 h (FIG. 12A Left and Center). These damaging effects were substantially averted by prior treatment of mouse skin for 3 days with daily doses of 100 nmol/cm² of sulforaphane delivered as a broccoli sprout extract (FIG. 12A Right). Skin myeloperoxidase (MPO) activity, which is localized in azurophilic granules of neutrophils and is a sensitive marker of inflammation intensity, increased in a dose-dependent manner upon UV radiation (>25-fold at 1,200 mJ/cm²) (FIG. 12B). Prior treatment with synthetic sulforaphane or with a broccoli sprout extract containing sulforaphane suppressed the increases of MPO protein and enzyme activity levels (FIGS. 12 C and D) and increased the specific activities of the prototypic phase 2 enzyme, NQO1 (FIG. 12E) in homogenates of the sulforaphane- or broccoli sprout extract-treated mouse skins. UV radiation depressed these inductions slightly (FIG. 1E). Topical treatment with either pure sulforaphane or broccoli sprout extract containing equivalent amount of sulforaphane showed quantitatively equivalent effects on the inductive increases in NQO1 and the inhibition of the UV radiation-dependent MPO activity.

This finding strongly supports the conclusions that: (i) both the phase 2 inducer activity and the protective effects against UV-mediated edema and inflammation (and probably other aspects of UV damage) provided by the broccoli sprout extract are entirely attributable to their sulforaphane content, and (ii) these effects do not arise from direct UV radiation absorption because sulforaphane has negligible absorption at 311 nm, whereas broccoli sprout extracts are aqueous plant extracts and are colored.

### Measurement of UV Erythema and Design of a Template for Treatment and Radiation

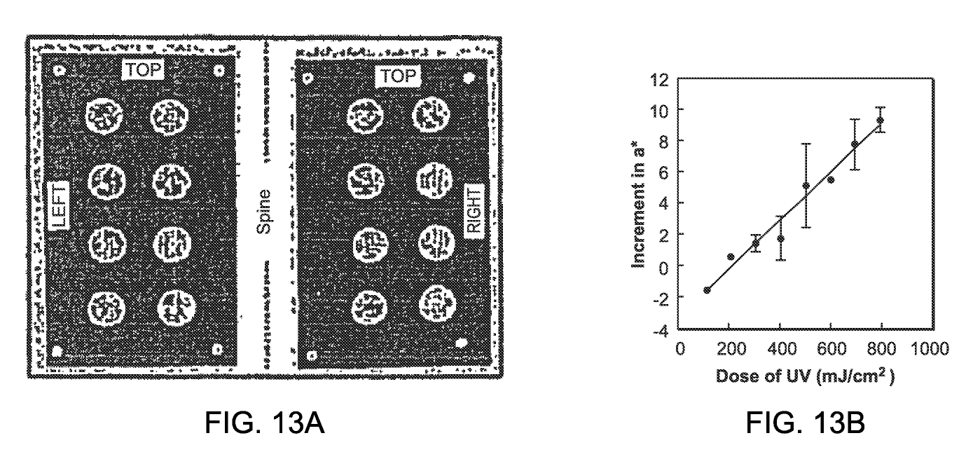

Translation of these findings from mice to humans required the development of highly quantitative and reproducible methods for evaluating UV-mediated damage of human skin. Erythema was used as a noninvasive biomarker. We designed a reusable, adhesive vinyl template that could be precisely positioned on the skin to make repetitive measurements of red reflectivity at exactly the same areas (spots) that were treated with standardized 311-nm doses of UV radiation and with potential protectors. Two 10×17 cm rectangular, opaque vinyl templates, each with four pairs of 2.0-cm diameter circular windows, were attached on successive days in precisely the same paraspinal region of the backs of volunteers (FIG. 13A). The windows could be occluded individually by easily removable vinyl shades (adhesive at the periphery, but non-adhesive over the windows), so that graded UV dosages could be delivered to the spots. Narrow-band UV (centered at 311 nm) was delivered in a Daavlin Full Body Phototherapy Cabinet with NB-UVB/TL01 lamps equipped with an integrated UVB dosimeter (BryanOH). The windows were used to produce either the same dose of UV to all windows or graded doses from 100-800 mJ/cm² to selected pairs of horizontally adjacent windows. Subjects were of skin phototypes 1 (always burns, never tans), 2 (always burns, sometimes tans) or 3 (sometimes burns, always tans). The erythema of each spot was quantified under standardized conditions with a chromometer (model CR-400; Konica Minolta) that determines the erythema index a*, a unit-less ratio of the intensities of the red reflectivity of the skin to the emission of a xenon arc flash adjusted for chromaticity along the green-red axis (Farr and Diffey, 1984; Diffey and Farr, 1991). The chromometer was calibrated with white and red tiles before each measurement session. Male and female volunteers (28-53 years of age) with skin type 1, 2, or 3 and no skin pathology were enrolled. The volunteers were asked to refrain from consuming cruciferous plants, including mustard, horseradish, wasabi, and condiments for one week before and during the study period. The volunteers were additionally instructed not to consume coffee or exercise before each study visit, which took place at 1300 h each day. No restrictions were placed on consumption of medications or dietary supplements. Subjects rested prone for 20 min in a temperature-controlled room (25°±2° C.) before measurements were made. Measurements were begun on each spot 20 seconds after allowing the skin to equilibrate under the weight of the chromometer (≈80 g). Eleven repetitive measurements were obtained on each spot in rapid succession (≈5 second intervals). The last eight values were used to calculate mean a* values and coefficients of variation (CVs) for each spot. All readings were obtained by a single trained operator.

The reproducibility of the measuring procedure was validated by obtaining mean a* values for all 16 spots of five subjects on 5 consecutive days (on the 4 days before and 24 h after UV exposure). These measurements established the following: (i) the last eight repetitive chromometer measurements made on the same 16 individual spots during the 4 days before UV radiation had a CV of 3.79 (SEM=0.19%; n=320 measurements), whereas 24 h after UV exposure the CV of the now higher a* values of 16 spots in the same five subjects was 2.26 (SEM=0.21%; n=80). Thus, repetitive measurements of the higher a* values of radiated spots could be determined with greater precision (P<0.0001); (ii) the initial mean a* values of the 16 spots in five individuals measured on 4 successive days before UV radiation were 4.52±1.89 (n=320). However, the variability of a* values among individual spots was considerably greater, ranging from 0.59-10.17. Although both the spot location and day of measurement significantly affected the basal a* values for a given individual, the CV, because of spot location alone, was 19.2%, whereas the CV from day to day was 6.2%. Thus, the differences in a* values among individual spots in a single subject were much larger than the variation between repetitive measurements on the same spot over time. This analysis of erythema index a* measurements led to the conclusion that each spot of any individual must be considered an independent observational unit; (iii) the increase in erythema resulting from UV radiation (Δa*) varied inversely with the initial value of a* before UV exposure (P=0.008), which is consistent with the view that lighter skin is more susceptible to erythema than darker skin; and (iv) the variation in UV radiation-induced erythema (Δa*) was random across the back, indicating no statistical bias to selecting vertically or horizontally adjacent control and treatment spots. On the basis of anatomical considerations (e.g., dermatomes and vasculature), horizontally adjacent spots were selected as treatment/control pairs.

### UV Radiation Dose-Response Curve

Having established a quantitative and reproducible system for assessing UV-mediated erythema, the relationship between UV dose and erythema response was examined in a 53-year-old single male with type 2 skin. Eight horizontally paired windows were exposed to UV radiation doses from 100-800 mJ/cm² in 100 ml/cm² increments, and a* measurements were made just before and 24 h after UV radiation. This range of UV radiation doses is widely used by dermatologists to determine the minimum erythema dose. The increments in a* values rose linearly with UV doses in this range (FIG. 13B), and there was reasonable agreement among duplicate areas even when the initial a* values of each spot were quite different. Therefore, increases in erythema were expressed as arithmetic increments in a* values for each individual spot, rather than the ratio of the a* value after UV radiation to that before UV radiation.

### Optimization of Sulforaphane Scheduling for Induction of NQO1 in Human Skin

To optimize the dosing schedule of sulforaphane for the present studies of protection of human skin against UV-induced erythema, 1.0-cm circular areas on the lower backs of three volunteers were treated with 5-μl aliquots of broccoli sprout extract containing 100 nmol of sulforaphane. The extract was applied at 24-h intervals on day 1, on day 3, on days 2 and 3, or on days 1, 2, and 3, and biopsies were obtained on day 4. Treatment on 3 successive days resulted in the largest induction of NQO1, with mean elevations of NQO1-specific activities of 2.19-fold (range 1.76-3.24). Therefore, in the following experiments, treatment with broccoli sprout extract was performed at 24-h intervals on 3 successive days before UV radiation.

### Protection Against UV Erythema Depends on Sulforaphane Dose

To optimize the protective doses of sulforaphane, one subject (male, age 53) received daily treatments with a range of doses of sulforaphane-containing broccoli sprout extract (containing 100, 200, 400, or 600 nmol of sulforaphane) on 3 successive days and was irradiated with 500 mJ/cm² of UV 24 h later. The increments in erythema a* values from before (mean of 4 days; 4.72±0.871) to 24 h. after radiation showed that sulforaphane treatment provided dose-dependent protection (FIGS. 3 A and B). The increase in erythema was inhibited by 26.3%, 44 4%, 57.6%, and 57.5% at daily doses of 100, 200, 400, or 600 nmol of sulforaphane per 2.0-cm diameters spot, respectively. This degree of protection by sulforaphane as a function of dose was in reasonable agreement with the dose-dependence of NQO1 induction as previously established in human skin (Dinkova-Kostova et al., 2007).

### Protection Against UV-Induced Erythema by Sulforaphane in Volunteers

To examine the protective effects of treatment with sulforaphane-containing broccoli sprout extract on UV dose-dependent erythema, the extracts were applied topically inside the 2.0 cm diameter circles of the vinyl template. Treated spots received the broccoli sprout extract in 25 μl of 80% acetone/20% water, and horizontally paired spots were treated with solvent only. Measurements of a* values were made on 5 consecutive days: 3 days before UV exposure, on the day of exposure immediately before UV radiation, and 24 h after exposure. Each subject was studied at eight doses of UV radiation (100-800 mJ/cm² in 100 mJ/cm² increments), and a* values were obtained for treated and control spots at each UV dose level. The a* measurements for each spot obtained on 4 successive days before UV radiation were averaged, and the means were used as the a*(pre-UV radiation) values. Pilot experiments showed that the increments in a* values (Δa*) after UV radiation [i.e., a*(post-UV radiation)−a*(pre-UV radiation)] were the most appropriate measurements of changes in skin erythema of individual spots. Because the response of individual subjects to a given dose of UV varied significantly (P<0 0001), as did the pre-UV radiation a* values, the protective effects of sulforaphane were expressed as the fractional reduction (%) in erythema upon treatment, thus providing a method of subject and skin region-specific normalization.

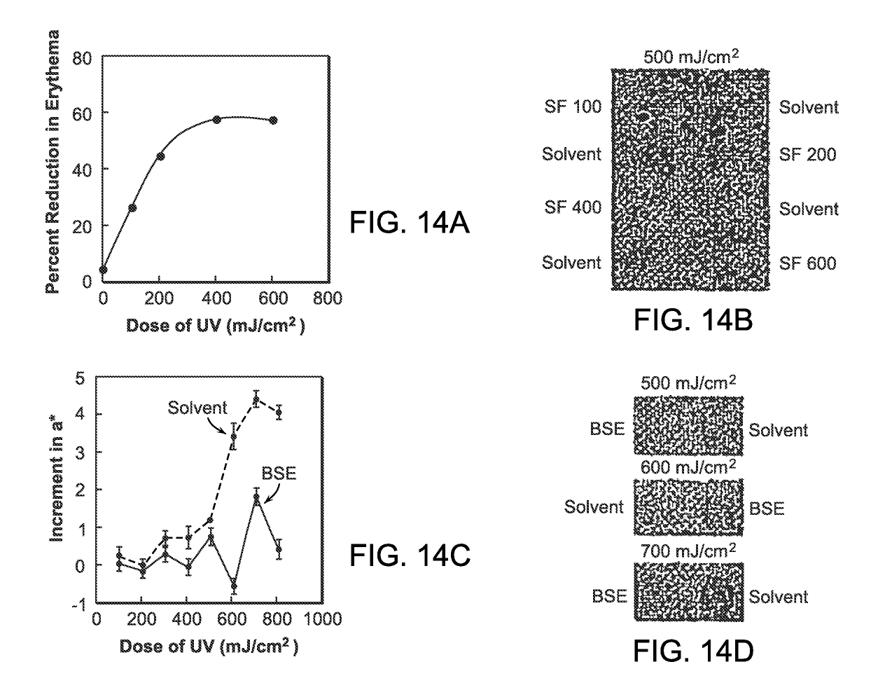

A typical result (FIGS. 14 C and D) established that sulforaphane treatment inhibited erythema development by 84.3%, 41.6%, and 89.4% at 600, 700, and 800 mJ/cm² doses of UV radiation, respectively, in spots that had been treated with broccoli sprout extract containing 200 nmol of sulforaphane on 3 successive days before radiation. Next, the protective effect of sulforaphane was examined in six volunteers who received broccoli sprout extract containing either 200 nmol (four subjects) or 400 nmol (two subjects) of sulforaphane on 3 successive days before radiation and were exposed to a range of eight doses of UV radiation (100-800 mJ/cm²). The responses at 100 and 200 mJ/cm² were excluded from the analysis (Table 1) because the increment in a* at these low UV doses was consistently smaller than their basal daily variations. The UV dose had an insignificant effect on the percent reduction of erythema (P=0.05), and trend analysis of the fractional reduction in erythema with respect to UV dose indicated no significant association (P=0.09). This finding suggests that the degree of protection is a relatively constant fraction of the erythema response irrespective of its magnitude. Therefore, sulforaphane probably protects against a relatively constant fraction of the multifactorial erythema response. To provide more power to the study, the data for all six subjects at six UV exposures were pooled. However, even without this restriction (n=35; one observation was not available) of the values, including those at which no erythema was observed, there was a highly significant effect of treatment (P<0.0001), and this finding was readily apparent visually. The pooled data revealed a mean level of protection for the six subjects across all six UV radiation doses (300-800 mJ/cm²) of 37.7% (SEM=5.7; n=35). This protection was highly significant [P<0.0001; 95% confidence interval (CI)=25-50%]. Moreover, when examined on an individual basis (i.e., across a row in Table 1), the mean protection of a given subject across all UV radiation doses was 37.7% (SEM=11.2; n=6), which also was significant (P=0.025; CI=11.8-64%). The a* measurements, confirmed by visual inspection, provided evidence that, although sulforaphane treatment inhibited UV-induced erythema in most observations (27 of 35 spots showed 8.7% or more protection), the response varied considerably both in individual subjects and among subjects.

TABLE 1
Effect of treatment with sulforaphane (broccoli sprout extract) on the erythema induced by UV radiation
Reduction in UVR-induced erythema at given UVB radiation dose, %

| | | | | | | | | | Mean | |
|---|---|---|---|---|---|---|---|---|---|---|
| | | Age, | 300 | 400 | 500 | 600 | 700 | 800 | reduction in | P |
| Subject | Sex | yr | mJ/cm² | mJ/cm² | m1/cm² | mJ/cm² | mJ/cm² | mJ/cm² | erythema, % | value |
| 1 | M | 53 | 66.8 | 32.3 | 33.1 | 16.6 | 48.8 | 32.1 | 38.3 | 0.0029 |
| 2 | F | 32 | 69.1 | −1.4 | −5.5 | 56.5 | 15.4 | 8.7 | 23.8 | 0.1220 |
| 3 | F | 28 | 30.1 | 1.7 | 1.5 | −5.7 | 22.2 | 0.4 | 8.37 | 0.2102 |
| 4 | M | 41 | 60.0 | 107.5 | 37.1 | 115.7 | 58.9 | 89.5 | 78.1 | 0.0016 |
| 5 | F | 29 | 52.0 | 72.5 | 87.0 | 64.5 | 26.7 | 20.9 | 53.9 | 0.0038 |
| 6 | M | 48 | N/A | 61.4 | 1.1 | 45.9 | 11.7 | −2.5 | 23.5 | 0.1390 |

| | |
|---|---|
| 37.7 ± 11.2 (±Sem) | 0.025 |

The six subjects (three men and three women) were studied under identical conditions over a 5-day period. The pairs of adhesive vinyl templates were applied in the same paraspinal positions on 4 successive days, at 24-h intervals, and erythema index (a*) values were determined with the chronometer on each of the 16 circular (2.0 cm diameter) windows at each session. The means of the last eight values of each set of measurements obtained on 4 days were averaged, and these means were assumed to be the a* values for each spot before UV radiation (Pre-UV radiation). Immediately after the last measurements, the subjects were exposed to a range of doses of UV (311 nm), such that the eight pairs of adjacent spots received 100-800 mJ/cm² in 100 mJ/cm² increments. Twenty-four hours after UV radiation, the chronometer a* measurements were repeated (Post-UV radiation). Only results for the 300-800 ml/cm²-UVR are shown. On the first 3 days, one of each pair of spots was treated with 25 μl of broccoli sprout extract containing 200-400 nmol sulforaphane (in 80% acetone/20% water), and the other received 25 μl of solvent only. The effects of treatment on UV radiation-induced erythema a* were derived from the change in a* values (Δa*), i.e., (a*Post-UV radiation−a*Pre-UV radiation) for broccoli sprout extract- and solvent-treated spots, and the percentage change expressed as follows: [(Δa* of treated spot/Δa* of control spot)×100]. The P values were calculated using a two-sided Student t test and represent the comparison between an individual subject's average percent reduction (i.e., across all UV radiation doses administered) and no protection (i.e., 0% reduction in erythema). For the purpose of the t test, the standard deviation associated with no protection (0% reduction) was assumed to be the same as that calculated for each individual. Consequently, in determining the significance of the mean percent reduction for all six subjects, the standard deviation associated with a no protection value (0%) was assumed to be equal to that of the individual subject responses.

Protection does not Depend on Absorption of UV Radiation

Three types of experiments provided convincing evidence that the protective effects of sulforaphane against UV radiation damage were not mediated by absorption of the incident UV radiation. (i) Application of a sunscreen preparation (≈10 mg per spot of Neutrogena Ultrasheer, Sun Protection Factor 55) for 3 days on the same schedule as the broccoli sprout extract and UV radiation (500 mJ/cm²) resulted in negligible protection (3.5%; mean of two observations) 24 h after the last application. Because volunteers were encouraged to retain their personal hygiene, it seems highly unlikely that a UV-absorbing effect could have persisted for 24 h or longer. (ii) Application of a broccoli sprout extract preparation delivering 400 nmol of unhydrolyzed glucoraphanin (the inactive glucosinolate precursor of sulforaphane) per spot provided negligible protection (4.7%; mean of two observations). These preparations were identical to the sulforaphane-containing broccoli sprout extract, except that the sulforaphane precursor had not been hydrolyzed by myrosinase. (iii) Treatment of one subject with broccoli sprout extract for 3 days according to the previous protocol, but delay of UV radiation for 48 or 72 h after the end of treatment, resulted in substantial continuing protection: 32.1% protection at 48 h and 10.3% at 72 h. These control experiments also shed light on the unique nature of a protective strategy that depends on transcriptional activation of a wide variety of enzymes. Thus, sulforaphane has a short tissue half-life (1-2 h), and yet its effects are clearly evident even 2-3 days after treatment because they depend on the synthesis of long-lived proteins. This long-lasting property has not been demonstrated for other topical skin protectors like sunscreens, melatonin, epigallocatechin gallate and carotenes (Baliga and Katiyar, 2006; Bangha et al., 1997). Moreover, experiments on mouse skin strongly suggest that the UV radiation protective effects of broccoli sprout extract are equivalent to those of an equivalent dose of pure sulforaphane. Because sulforaphane absorbs UV maximally near 240 nm and is almost transparent at 311 nm, this compound is unlikely to be decomposed or absorbed by UV radiation at 311 nm, in contrast to some of the other topical protective agents.

Abbreviations: COX-2, cyclooxygenase 2; GSH, glutathione; γ-IFN, interferon γ; iNOS, inducible nitric oxide synthase; LPS, lipopolysaccharide; NQO1, NAD(P)H-quinone acceptor oxidoreductase, also designated quinone reductase.

### REFERENCES

1. Afaq, F. and Mukhtar, H. (2001) Effects of solar radiation on cutaneous detoxification pathways. J. Photochem. Photobiol, 63: 61-69.
2. Alam, M. and Ratner, D. (2001) Cutaneous squamous-cell carcinoma. N. Engl. J. Med., 344, 975-983.
3. Anderson, M. E. (1985) Determination of glutathione and glutathione disulfide in biological samples. Methods Enzymol., 113, 548-555.
4. Asher, G., Lotem, J., Cohen, B., Sachs, L. and Shaul, Y. (2001) Regulation of p53 stability and p53-dependent apoptosis by NADH quinone oxidoreductase 1. Proc. Natl. Acad. Sci. USA, 98, 1188-1193.
5. Baglia, M. and Katiyar, S. K. 2006. Chemoprevention of photocarcinogenesis by selected dietary botanicals. Photochem. Photobiol. Sci. 5: 243-253.
6. Bangha, E., Elsner, P. and Kistler, G. S. 1997. Suppression of UV-induced erythema by topical treatment with melatonin (N-acetyl-5-methoxytryptamine). A dose response study. Dermatology 195: 248-252.
7. Black, H. S., and Chan, J. T. (1975) Suppression of ultraviolet light-induced tumor formation by dietary antioxidants. J. Invest. Dermatol., 65, 412-414.
8. Black, H. S., Chan, J. T. and Brown, G. E. (1978) Effects of dietary constituents on ultraviolet light-mediated carcinogenesis. Cancer Res., 38, 1384-1387.
9. Braun, S., Hanselmann, C., Gassmann, M. G., auf dem Keller, U., Born-Berclaz, C., Chan, K., Kan, Y. W., and Werner, S. 2002. Nrf2 transcription factor, a novel target of keratinocyte growth factor action which regulates gene expression and inflammation in the healing skin wound. Mol Cell Biol 22(15):5492-5505.
10. Chen C, Yu R, Owuor E D, Kong A N. (2000) Activation of antioxidant-response element (ARE), mitogen-activated protein kinases (MAPKs) and caspases by major green tea polyphenol components during cell survival and death. Arch. Pharm. Res., 23, 605-612.
11. Choi, S. and Singh, S. V. (2005) Bax and Bak are required for apoptosis induction by sulforaphane, a cruciferous vegetable-derived cancer chemopreventive agent. Cancer Res., 65, 2035-2043.
12. Chung, F. L., Conaway, C. C., Rao, C. V. and Reddy, B. S. (2000) Chemoprevention of colonic aberrant crypt foci in Fischer rats by sulforaphane and phenethyl isothiocyanate. Carcinogenesis, 21, 2287-2291.
13. Clairmont, A., Sies, H., Ramachandran, S., Lear, J. T., Smith, A. G., Bowers, B., Jones, P. W., Fryer, A. A. and Strange, R. C. (1999) Association of NAD(P)H: quinone oxidoreductase (NQO1) null with numbers of basal cell carcinomas: use of a multivariate model to rank the relative importance of this polymorphism and those at other relevant loci. Carcinogenesis, 20, 1235-1240.
14. Diepgen, T. L. and Mahler, V. (2002) The epidemiology of skin cancer. Br. J. Dermatol., 146, 1-6.
15. Diffey, B. L. and Farr, P. M. 1991. Quantitative aspects of ultraviolet erythema. Clin. Phys. Physiol. Meas. 12: 311-325.
16. Dinkova-Kostova A T, Fahey J W, Wade K L, Jenkins S N, Shapiro T A, Fuchs E J, Kerns M L, Talalay P. 2007. Induction of the Phase 2 Response in Mouse and Human Skin by Sulforaphane-containing Broccoli Sprout Extracts. Cancer Epidemiol Biomarkers Prev. 16(4): 847-51.
17. Dinkova-Kostova, A. T., Liby, K. T., Stephenson, K. K., Holtzclaw, W. D., Gao, X., Suh, N., Williams, C., Risingsong, R., Honda, T., Gribble, G. W., Sporn, M. B. and Talalay, P. (2005) Extremely potent triterpenoid inducers of the phase 2 response: correlations of protection against oxidant and inflammatory stress. Proc. Natl. Acad. Sci. USA, 102, 4584-4589.
18. Ehrhart, J. C., Gosselet, F. P., Culerrier, R. M. and Sarasin, A. (2003) UVB-induced mutations in human key gatekeeper genes governing signalling pathways and consequences for skin tumourigenesis. Photochem. Photobiol. Sci., 2, 825-834.
19. Fahey, J. W., Dinkova-Kostova. A. T., Stephenson, K. K. and Talalay, P. 2004. The “Prochaska” microtiter plate bioassay for inducers of NQ01. Methods Enzymol., 382, 243-258.
20. Fahey, J. W., Haristoy, X., Dolan, P. M., Kensler, T. W., Scholtus, I, Stephenson, K. K., Talalay, P. and Lozniewski, A. (2002) Sulforaphane inhibits extracellular, intracellular, and antibiotic-resistant strains of Helicobacter pylori and prevents benzo[a]pyrene-induced stomach tumors. Proc. Natl. Acad. Sci. USA, 99, 7610-7615.
21. Fahey, J. W. and Talalay, P. 1999. Antioxidant functions of sulforaphane: a potent inducer of Phase II detoxication enzymes. Food Chem Toxicol 37(9-10): 973-979.
22. Fahey, J. W., Zhang, Y. and Talalay, P. (1997) Broccoli sprouts: an exceptionally rich source of inducers of enzymes that protect against chemical carcinogens. Proc. Natl. Acad. Sci. USA, 94, 10367-10372.
23. Farr, P. M. and Diffey, B. L. 1984. Quantitative studies on cutaneous erythema induced by ultraviolet radiation. Br. J. Dermatol. 111: 673-682.
24. Gamet-Payrastre, L., Li, P., Lumeau, S., Cassar, G., Dupont, M. A., Chevolleau, S., Gasc, N., Tulliez, J. and Tercé, F. (2000) Sulforaphane, a naturally occurring isothiocyanate, induces cell cycle arrest and apoptosis in HT29 human colon cancer cells. Cancer Res., 60, 1426-1433.
25. Gao, X., Dinkova-Kostova, A. T. and Talalay, P. (2001) Powerful and prolonged protection of human retinal pigment epithelial cells, keratinocytes, and mouse leukemia cells against oxidative damage: the indirect antioxidant effects of sulforaphane. Proc. Natl. Acad. Sci. USA, 98,15221-15226.
26. Gao, X. and Talalay, P. (2004) Induction of phase 2 genes by sulforaphane protects retinal pigment epithelial cells against photooxidative damage. Proc. Natl. Acad. Sci. USA, 2004 101, 10446-10451.
27. Gills J J, Jeffery E H, Matusheski N V, Moon R C, Lantvit D D, Pezzuto J M. 2006. Sulforaphane prevents mouse skin tumorigenesis during the stage of promotion. Cancer Lett. 236(1):72-9.
28. Gross C L, Nealley E W, Nipwoda M T, Smith W J. 2006. Pretreatment of human epidermal keratinocytes with D,L-sulforaphane protects against sulfur mustard cytotoxicity. Cutan Ocul Toxicol. 25(3):155-63.
29. Haristoy X, Angioi-Duprez K, Duprez A, Lozniewski A. 2003. Efficacy of sulforaphane in eradicating Helicobacter pylori in human gastric xenografts implanted in nude mice. Antimicrob Agents Chemother. 47(12):3982-4.
30. Hecht, S. S. 1995. Chemoprevention by isothiocyanates. J Cell Biochem Suppl 22: 195-209.
31. Heiss, E., Herhaus, C., Klimo, K., Bartsch, H. and Gerhäuser, C. (2001) Nuclear factor kappa B is a molecular target for sulforaphane-mediated anti-inflammatory mechanisms. J. Biol. Chem., 276, 32008-32015.
32. Hotlzclaw, W. D., Dinkova-Kostova, A. T., Talalay, P. 2004. Protection against electrophile and oxidative stress by induction of phase 2 genes: The quest for the elusive sensor that responds to inducers. Adv Enzyme Regul. 44: 335-367.
33. Hussein, M. R. (2005) Ultraviolet radiation and skin cancer: molecular mechanisms. J. Cutan. Pathol., 32, 191-205.
34. Iida K, Itoh K, Kumagai Y, Oyasu R, Hattori K, Kawai K, Shimazui T, Akaza H, Yamamoto M. 2004. Nrf2 is essential for the chemopreventive efficacy of oltipraz against urinary bladder carcinogenesis. Cancer Res. 64: 6424-31.
35. Iskander, K., Gaikwad, A., Paquet, M., Long II, D. J., Brayton, C., Barrios, R. and Jaiswal, A, K. (2005) Lower induction of p53 and decreased apoptosis in NQO1-null mice lead to increased sensitivity to chemical-induced skin carcinogenesis. Cancer Res., 65, 2054-2058.
36. Juurlink, B. H. (2001) Therapeutic potential of dietary phase 2 enzyme inducers in ameliorating diseases that have an underlying inflammatory component. Can. J. Physiol. Pharmacol., 79, 266-282.
37. Katiyar, S. K., Afaq, F., Perez, A. and Mukhtar, H. (2001) Green tea polyphenol (−)-epigallo-catechin-3-gallate treatment of human skin inhibits ultraviolet radiation-induced oxidative stress. Carcinogenesis, 22, 287-294.
38. Katiyar, S. K., Matsui, M. S, and Mukhtar, H. (2000) Kinetics of UV light-induced cyclobutane pyrimidine dimers in human skin in vivo: an immunohistochemical analysis of both epidermis and dermis. Photochem. Photobiol., 72, 788-793.
39. Keum Y S, Yu S, Chang P P, Yuan X, Kim J H, Xu C, Han J, Agarwal A, Kong A N. 2006. Mechanism of Action of Sulforaphane: Inhibition of p38 Mitogen-Activated Protein Kinase Isoforms Contributing to the Induction of Antioxidant Response Element-Mediated Heme Oxygenase-1 in Human Hepatoma HepG2 Cells. Cancer Res. 66(17):8804-13.
40. Killeen M E, Englert J A, Stolz D B, Song M, Han Y, Delude R L, Kellum J A, Fink M P. 2006. The phase 2 enzyme inducers ethacrynic acid, DL-sulforaphane, and oltipraz inhibit lipopolysaccharide-induced high-mobility group box 1 secretion by RAW 264.7 cells. J Pharmacol Exp Ther. 316(3):1070-9.
41. Kozumbo, W. J., Seed, J. L. and Kensler, T. W. (1983) Inhibition by 2(3)-tert-butyl-4-hydroxyanisole and other antioxidants of epidermal ornithine decarboxylase activity induced by 12-O-tetradecanoylphorbol-13-acetate. Cancer Res., 43, 2555-2559.
42. LeBel, C. P., Ischiropoulos, H. and Bondy, S. C. (1992) Evaluation of the probe 2′,7′-dichlorofluorescin as an indicator of reactive oxygen species formation and oxidative stress. Chem. Res. Toxicol., 5, 227-231.
43. Li, J., Lee, J. M. and Johnson, J. A. (2002) Microarray analysis reveals an antioxidant responsive element-driven gene set involved in conferring protection from an oxidative stress-induced apoptosis in IMR-32 cells. J. Biol. Chem., 277, 388-394.
44. Lu, Y. P., Lou, Y. R., Lin, Y., Shih, W. J., Huang, M. T., Yang, C. S. and Conney, A. H. 2001. Inhibitory effects of orally administered green tea, black tea, and caffeine on skin carcinogenesis in mice previously treated with ultraviolet B light (high-risk mice): relationship to decreased tissue fat. Cancer Res., 61, 5002-5009.
45. Lu, Y. P., Lou, Y. R., Yen, P., Mitchell, D., Huang, M. T. and Conney, A. H. (1999) Time course for early adaptive responses to ultraviolet B light in the epidermis of SKH-1 mice. Cancer Res., 59, 4591-4602.
46. Lu, Y. P., Lou, Y. R., Xie, J. G., Peng, Q. Y., Liao, J., Yang, C. S., Huang, M. T. and Conney, A. H. 2002. Topical applications of caffeine or (−)-epigallocatechin gallate (EGCG) inhibit carcinogenesis and selectively increase apoptosis in UVB-induced skin tumors in mice. Proc. Natl. Acad. Sci. USA, 99, 12455-12460.
47. Merk, H., Jugert, F., Bonnekoh, B. and Mahrle, G. (1991) Induction and inhibition of NAD(P)H: quinone reductase in murine and human skin. Skin Pharmacol., 4, 183-190.
48. Motohashi H, and Yamamoto M. 2004. Nrf2-Keap1 defines a physiologically important stress response mechanism. Trends Mol. Med. 10(11):549-57.
49. Myzak, M. C., Karplus, P. A., Chung, F. L. and Dashwood, R. H. (2004) A novel mechanism of chemoprotection by sulforaphane: inhibition of histone deacetylase. Cancer Res., 64, 5767-5774.
50. Nishigori, C., Yarosh, D. B., Donawho, C. and Kripke, M. L. (1996) The immune system in ultraviolet carcinogenesis. J. Investig. Dermatol. Symp. Proc., 1, 143-146.
51. Peak, M. J., Peak, J. G. and Carnes, B. A. (1987) Induction of direct and indirect single-strand breaks in human cell DNA by far- and near-ultraviolet radiations: action spectrum and mechanisms. Photochem. Photobiol., 45, 381-387.
52. Pinnell, S. R. (2003) Cutaneous photodamage, oxidative stress, and topical antioxidant protection. J. Am. Acad. Dermatol., 48, 1-19.
53. Pledgie-Tracy A, Sobolewski M D, Davidson N E. 2007. Sulforaphane induces cell type-specific apoptosis in human breast cancer cell lines. Mol Cancer Ther. 6(3):1013-21.
54. Prochaska, H. J. and Santamaria, A. B. (1988) Direct measurement of NAD(P)H: quinone reductase from cells cultured in microtiter wells: a screening assay for anticarcinogenic enzyme inducers. Anal. Biochem., 169, 328-336.
55. Record, I. R. and Dreosti, I. E. (1998) Protection by black tea and green tea against UVB and UVA+B induced skin cancer in hairless mice. Mutat. Res., 422, 191-199.
56. Richie, J. P. Jr., Skowronski, L., Abraham, P. and Leutzinger, Y. (1996) Blood glutathione concentrations in a large-scale human study. Clin. Chem., 42, 64-70.
57. Ross, D. (2004) Quinone reductases multitasking in the metabolic world. Drug Metab. Rev., 36, 639-654.
58. Sander, C. S., Chang, H., Hamm, F., Elsner, P. and Thiele, J. J. (2004) Role of oxidative stress and the antioxidant network in cutaneous carcinogenesis. Int. J. Dermatol., 43, 326-335.
59. Sarasin, A. (1999) The molecular pathways of ultraviolet-induced carcinogenesis. Mutat. Res., 428, 5-10.
60. Setlow, R. B. and Carrier, W. L. (1966) Pyrimidine dimers in ultraviolet-irradiated DNA's. J. Mol. Biol., 17, 237-254.
61. Shapiro, T. A., Fahey, J. W., Wade, K. L., Stephenson, K. K. and Talalay, P. (2001) Disposition of chemoprotective glucosinolates and isothiocyanates of broccoli sprouts. Cancer Epidemiol. Biomark. Prevent., 10, 501-508.
62. Slaga, T. J. and Bracken, W. M. (1977) The effects of antioxidants on skin tumor initiation and aryl hydrocarbon hydroxylase. Cancer Res., 37, 1631-1635.
63. Suh, N., Honda, T., Finlay, H. J., Barchowsky, A., Williams, C., Benoit, N. E., Xie, Q. W., Nathan, C., Gribble, G. W. and Sporn, M. B. (1998) Novel triterpenoids suppress inducible nitric oxide synthase (iNOS) and inducible cyclooxygenase (COX-2) in mouse macrophages. Cancer Res., 58, 717-723.
64. Talalay, P. (1989) Mechanisms of induction of enzymes that protect against chemical carcinogenesis. Adv. Enzyme Regul., 28, 237-250.
65. Talalay, P. (2000) Chemoprotection against cancer by induction of phase 2 enzymes. Biofactors, 12, 5-11.
66. Talalay, P., Batzinger, R. P., Benson, A. M., Bueding, E. and Cha, Y. N. (1979) Biochemical studies on the mechanisms by which dietary antioxidants suppress mutagenic activity. Adv. Enzyme. Regul., 17, 23-36.
67. Troyer, J. K., Stephenson, K. K. and Fahey, J. W. (2001) Analysis of glucosinolates from broccoli and other cruciferous vegetables by hydrophilic interaction liquid chromatography. J. Chromatogr. A, 919, 299-304.
68. Wei, H., Zhang, X., Wang, Y. and Lebwohl, M. (2002) Inhibition of ultraviolet light-induced oxidative events in the skin and internal organs of hairless mice by isoflavone genistein. Cancer Lett., 185, 21-29.
69. Wu, L., Noyan Ashraf, M. H., Facci, M., Wang, R., Paterson, P. G., Ferrie, A. and Juurlink, B. H. (2004) Dietary approach to attenuate oxidative stress, hypertension, and inflammation in the cardiovascular system. Proc. Natl. Acad. Sci. USA, 101, 7094-7099.
70. Xu C, Shen G, Yuan X, Kim J H, Gopalkrishnan A, Keum Y S, Nair S, Kong A N. 2006. ERK and JNK signaling pathways are involved in the regulation of activator protein 1 and cell death elicited by three isothiocyanates in human prostate cancer PC-3 cells. Carcinogenesis. 27(3):437-45.
71. Ye, L. and Zhang, Y. (2001) Total intracellular accumulation levels of dietary isothiocyanates determine their activity in elevation of cellular glutathione and induction of Phase 2 detoxification enzymes. Carcinogenesis, 22, 1987-1992.
72. Zhang, Y., Kensler, T. W., Cho, C. G., Posner, G. H. and Talalay, P. (1994) Anticarcinogenic activities of sulforaphane and structurally related synthetic norbornyl isothiocyanates. Proc. Natl. Acad. Sci. USA, 91, 3147-3150.
73. Zhang, Y. and Talalay, P. 1994. Anticarcinogenic activities of organic isothiocyanates: chemistry and mechanisms. Cancer Res 54(7 Supply: 1976s-1981s.
74. Zhang, Y., Talalay, P., Cho, C. G., and Posner, G. H. 1992. A major inducer of anticarcinogenic protective enzymes from broccoli: isolation and elucidation of structure. Proc Natl Acad Sci USA 89(6): 2399-2403.
75. Zhang, Y., Wade, K. L., Prestera, T. and Talalay, P. (1996) Quantitative determination of isothiocyanates, dithiocarbamates, carbon disulfide, and related thiocarbonyl compounds by cyclocondensation with 1,2-benzenedithiol. Anal. Biochem., 239, 160-167.
76. Thu M, Zhang Y, Cooper S, Sikorski E, Rohwer J, Bowden G T. 2004. Phase II enzyme inducer, sulforaphane, inhibits UVB-induced AP-1 activation in human keratinocytes by a novel mechanism. Mol. Carcinog. 41(3):179-86.
77. Zoete, V., Rougėe, M., Dinkova-Kostova, A. T., Talalay, P. and Bensasson, R. V. (2004) Redox ranking of inducers of a cancer-protective enzyme via the energy of their highest occupied molecular orbital. Free Radic. Biol. Med., 36, 1418-1423.

What is claimed is:

1. A method of protecting the skin and mucous membranes from an undesirable side effect of ionizing radiations in a patient undergoing ionizing radiation therapy comprising topically administering to the area of the patient's body exposed to ionizing radiation and surrounding areas a composition comprising a therapeutically effective amount of an Nrf2 inducer.

2. The method of claim 1, wherein the undesirable side effect is selected from the group consisting of acute erythema, skin irritation, inflammation, edema, desquamation, necrosis of the skin, soreness, ulceration in the mouth, pain, fibrosis, telangiectasia, xerostomia, xerophthalmia, dryness of the vaginal mucosa, melanoma, breast cancer, stomach cancer, lung cancer and thyroid disorders.

3. The method of claim 1, wherein the Nrf2 inducer is a phase II enzyme inducer.

4. The method of claim 3, wherein the phase II inducer is an isothiocyanate.

5. The method of claim 4, wherein the phase II enzyme inducer is sulforaphane or a sulforaphane synthetic analogue.

6. The method of claim 5, wherein the sulforaphane synthetic analogue is selected from the group consisting of 6-isothiocyanato-2-hexanone, exo-2-acetyl-6-isothiocyanatonorbornane, exo-2-isothiocyanato-6-methylsulfonylnorbornane, 6-isothiocyanato-2-hexanol, 1-isothiocyanato-4-dimethylphosphonylbutane, exo-2-(1′-hydroxyethyl)-5-isothiocyanatonorbornane, exo-2-acetyl-5-isothiocyanatonorbornane, 1-isothiocyanato-5-methylsulfonylpentane, cis-3-(methylsulfonyl)cyclohexylmethylisothiocyanate and trans-3-(methylsulfonyl)cyclohexylmethylisothiocyanate.

7. The method of claim 3, wherein the phase II inducer is a glucosinolate.

8. The method of claim 1, wherein the composition is administered to the patient prior to, during or after ionizing radiation therapy.

9. The method of claim 1, wherein the amount of Nrf2 inducer in the composition topically administered to the patient is from about 100 nmol to about 1 μmol/cm².

10. The method of claim 1, wherein the composition comprising the Nrf2 inducer is a topical preparation selected from the group consisting of ointment, cream, emulsion, lotion, gel and sunscreen.

11. The method of claim 1, wherein the patient is a mammal.

12. The method of claim 11, wherein the mammal is a human.

13. A composition for topical application to the skin comprising a therapeutically effective amount of an Nrf2 inducer and a vehicle selected from the group consisting of jojoba oil and evening primrose oil.

14. The composition of claim 13 in the form of ointment, cream, emulsion, lotion, gel or sunscreen.

15. The composition of claim 13, wherein the Nrf2 inducer is a phase II enzyme inducer.

16. The composition of claim 15, wherein the phase II inducer is an isothiocyanate.

17. The composition of claim 16, wherein the phase II enzyme inducer is sulforaphane or a sulforaphane synthetic analogue.

18. The composition of claim 17, wherein the sulforaphane synthetic analogue is selected from the group consisting of 6-isothiocyanato-2-hexanone, exo-2-acetyl-6-isothiocyanatonorbornane, exo-2-isothiocyanato-6-methylsulfonylnorbornane, 6-isothiocyanato-2-hexanol, 1-isothiocyanato-4-dimethylphosphonylbutane, exo-2-(1′-hydroxyethyl)-5-isothiocyanatonorbornane, exo-2-acetyl-5-isothiocyanatonorbornane, 1-isothiocyanato-5-methylsulfonylpentane, cis-3-(methylsulfonyl)cyclohexylmethylisothiocyanate and trans-3-(methylsulfonyl)cyclohexylmethylisothiocyanate.

19. The composition of claim 15, wherein the Nrf2 inducer is a glucosinolate.

20. The composition of claim 13, wherein the amount of Nrf2 inducer is from about 100 nmol to about 1 μmol/cm².
